SEQUENCE LISTING (4 sequences)

SEQ ID NO: 1 - protein, 15 aa, homo sapiens
IAQELRRIGDEFNAY

SEQ ID NO: 2 - protein, 15 aa, mus musculus
IAQELRRIGDEFNET

SEQ ID NO: 3 - protein, 16 aa, homo sapiens
GQVGRQLAIIGDDINR

SEQ ID NO: 4 - protein, 25 aa, homo sapiens
DMRPEIWIAQELRRIGDEFNAYYAR

(19) United States
(12) Patent Application Publication
(10) Pub. No.: US 2013/0295185 A1
(43) Pub. Date: Nov. 7, 2013

(54) COMPOUNDS AND METHODS FOR INDUCING APOPTOSIS IN CANCER CELLS USING A BH3 ALPHA-HELICAL MIMETIC

(71) Applicants: H. Lee Moffitt Cancer Center and Research Institute, Inc., Tampa, FL (US); Intezyne Technologies, Inc., Tampa, FL (US); Yale University, New Haven, CT (US)

(72) Inventors: Said M. Sebti, Tampa, FL (US); Andrew D. Hamilton, Oxfordshire (GB); Kevin Sill, Tampa, FL (US); Adam Carie, Ruskin, FL (US)

(21) Appl. No.: 13/940,967

(22) Filed: Jul. 12, 2013

Related U.S. Application Data
(63) Continuation of application No. PCT/US12/21049, filed on Jan. 12, 2012.
(60) Provisional application No. 61/431,960, filed on Jan. 12, 2011.

(51) Int. Cl. C07C 59/70; A61K 31/194; A61K 45/06; A61K 9/107

(52) U.S. Cl. CPC C07C 59/70; A61K 9/107; A61K 31/194; A61K 45/06
USPC 424/489; 562/468; 514/571; 514/64; 514/423; 514/19.3; 435/375

(57) ABSTRACT

A novel BH3 α-helical mimetic, BH3-M6, which binds to Bcl-X_{L} and prevents its binding to fluorescently-labeled Bak-BH3 peptide in vitro with an IC₅₀ value of 734 nM is presented herein. BH3-M6 is a pan-Bcl-2 antagonist that inhibits the binding of Bcl-X_{L}, Bcl-2 and Mcl-1 to multi-domain Bax or Bak, or BH3-only Bim or Bad in a cell-free system and in intact human cancer cells, freeing up pro-apoptotic proteins to induce apoptosis. BH3-M6-induced apoptosis is caspase- and Bax-dependent. Furthermore, human cancer cells with high Bcl-2 or Bcl-X_{L} levels are more sensitive to BH3-M6-induced cell death, suggesting that this compound can overcome drug resistance due to Bcl-2 or Bcl-X_{L} overexpression. The pan-Bcl-2 inhibitor BH3-M6 may be encapsulated in a micelle to provide a more bioavailable therapeutic agent. Specifically, the BH3-M6 compound may be encapsulated within a micelle comprising a multiblock copolymer according to the methods described herein.

### CROSS REFERENCE TO RELATED APPLICATIONS

This application is a continuation of prior filed International Application, Serial Number PCT/US2012/021049 filed Jan. 12, 2012, which claims priority to U.S. Provisional Application No. 61/431,960 entitled “Method of Inducing Apoptosis Using a BH3 Alpha-Helical Mimetic”, filed Jan. 12, 2011, the contents of which are hereby incorporated by reference into this disclosure.

### GOVERNMENTAL SUPPORT

This invention was made with Government support under Grant Nos. CA118210 and GM069850 awarded by the National Institutes of Health (NIH). The Government has certain rights in the invention.

### FIELD OF INVENTION

This invention relates to treatment of cancer. Specifically, the invention provides compounds and methods of treating cancer by inducing apoptosis in cancer cells.

### BACKGROUND OF THE INVENTION

Apoptosis, a form of programmed cell death, is a highly conserved process in all multicellular organisms and is essential for embryonic development and adult tissue homeostasis. Deregulation of apoptosis contributes to several diseases including cancer. (Green, D. R., and Evan, G. I. (2002) Cancer Cell 1, 19-30) Apoptosis is primarily controlled by two major pathways, namely the death receptor (extrinsic) and the mitochondrial (intrinsic) pathways. (Wei, M. C., Zong, W. X., Cheng, E. H., Lindsten, T., Panoutsakopoulou, V., Ross, A. J., Roth, K. A., MacGregor, G. R., Thompson, C. B., and Korsmeyer, S. J. (2001) Science 292, 727-730) The former is mediated by members of the tumor necrosis factor (TNF) receptor superfamily, while the latter largely depends on multiple Bcl-2 family proteins, which affect the integrity of the mitochondrial outer membrane (MOM). (Korsmeyer, S. J. (1999) Cancer Res 59, 1693s-1700s) Both pathways converge on common cysteine proteases of the caspase family, which are responsible for the execution of apoptosis. (Hengartner, M. O. (2000) Nature 407, 770-776)

The Bcl-2 family consists of anti-apoptotic and pro-apoptotic proteins. Anti-apoptotic proteins, such as Bcl-2, Bcl-X_{L}, Bcl-w, Mcl-1, and Bfl-1 (Bcl-2A1) contain four Bcl-2 homology (BH) domains, while the pro-apoptotic members are divided into proteins with three BH domains BH1-BH3 (Bax, Bak, and Bok), and proteins with only a BH3 domain (e.g., Bim, Bad, Bik, Bmf, Bid, Noxa, and Puma). (Adams, J. M., and Cory, S. (2007) Oncogene 26, 1324-1337) Multi-domain pro-apoptotic proteins Bax and Bak are absolutely required for apoptosis. (Wei, M. C., Zong, W. X., Cheng, E. H., Lindsten, T., Panoutsakopoulou, V., Ross, A. J., Roth, K. A., MacGregor, G. R., Thompson, C. B., and Korsmeyer, S. J. (2001) Science 292, 727-730) In response to cellular stress, they induce the release from mitochondria of apoptogenic factors such as cytochrome c, which then cooperate with APAF-1 to induce caspase-9 activation, followed by caspase-mediated apoptosis. (Chipuk, J. E., and Green, D. R. (2008) Trends Cell Biol 18, 157-164) BH3-only proteins act upstream of Bax and Bak and are important for the initiation of apoptosis. Importantly, the BH3 domain is essential for the killing function of pro-apoptotic proteins. (Willis, S, N., and Adams, J. M. (2005) Curr Opin Cell Biol 17, 617-625)

An important feature of the Bcl-2 proteins is that they can homo- and heterodimerize, giving rise to three competing, but not necessarily exclusive models that could explain how the balance between pro- and anti-apoptotic proteins regulates apoptosis. (Willis, S. N., and Adams, J. M. (2005) Curr Opin Cell Biol 17, 617-625) For instance, upon receiving an apoptotic signal, BH3-only proteins directly or indirectly induce Bax and Bak activation and homo-oligomerization in the MOM, which is thought to be responsible for MOM permeabilization, resulting in the release of cytochrome c and the initiation of intrinsic apoptosis. However, activated Bax and Bak still can be kept in check by binding to anti-apoptotic Bcl-2 proteins. (Yin, X. M., Oltvai, Z. N., and Korsmeyer, S. J. (1994) Nature 369, 321-323; Chittenden, T., Harrington, E. A., O'Connor, R., Flemington, C., Lutz, R. J., Evan, G. I., and Guild, B. C. (1995) Nature 374, 733-736; Lovell, J. F., Billen, L. P., Bindner, S., Shamas-Din, A., Fradin, C., Leber, B., and Andrews, D. W. (2008) Cell 135, 1074-1084)

X-ray diffraction and nuclear magnetic resonance (NMR) studies have shown that the amphipathic α-helices of pro-apoptotic proteins such as Bak or Bad BH3 domains fit into a hydrophobic pocket formed by the BH1, BH2, and BH3 domains of Bcl-2, Bcl-X_{L} and Mcl-1. (Pellecchia, M., and Reed, J. C. (2004) Curr Pharm Des 10, 1387-1398)

When BH3-only proteins bind to anti-apoptotic Bcl-2 proteins, multi-domain proteins Bak or Bax become free to induce apoptosis. (Yang, E., Zha, J., Jockel, J., Boise, L. H., Thompson, C. B., and Korsmeyer, S. J. (1995) Cell 80, 285-291) BH3-only proteins Bim, Bid and Puma can engage all Bcl-2 anti-apoptotic proteins, and are thus the most efficient killers. (Willis, S. N., and Adams, J. M. (2005) Curr Opin Cell Biol 17, 617-625) This mechanism is known as the indirect activation model. (Chipuk, J. E., and Green, D. R. (2008) Trends Cell Biol 18, 157-164; Gavathiotis, E., Suzuki, M., Davis, M. L., Pitter, K., Bird, G. H., Katz, S. G., Tu, H.-C., Kim, H., Cheng, E. H.-Y., Tjandra, N., and Walensky, L. D. (2008) Nature 455, 1076-1081) Additionally, certain BH3-only proteins (t-Bid, Bim and potentially Puma) can directly activate Bax, and this is known as the direct activation model. (Yamaguchi, H., and Wang, H. G. (2002) J Biol Chem 277, 41604-41612) Thus, it was recently demonstrated that the Bim-derived BH3 α-helix activates Bax through binding to a site that is distinct from the hydrophobic pocket of the anti-apoptotic proteins. (Gavathiotis, E., Suzuki, M., Davis, M. L., Pitter, K., Bird, G. H., Katz, S. G., Tu, H.-C., Kim, H., Cheng, E. H.-Y., Tjandra, N., and Walensky, L. D. (2008) Nature 455, 1076-1081) A third model suggests that cells can be “poised for death” but survive if their anti-apoptotic proteins sequester sufficient amounts of pro-apoptotic BH3-only proteins. (Certo, M., Del Gaizo Moore, V., Nishino, M., Wei, G., Korsmeyer, S., Armstrong, S. A., and Letai, A. (2006) Cancer Cell 9, 351-365)

The fact that overexpression of anti-apoptotic Bcl-2 proteins contributes to oncogenesis and drug resistance prompted the search for antagonists of these proteins as novel anti-cancer drugs. (Adams, J. M., and Cory, S. (2007) Oncogene 26, 1324-1337; Reed, J. C., Miyashita, T., Takayama, S., Wang, H. G., Sato, T., Krajewski, S., Aime-Sempe, C., Bodrug, S., Kitada, S., and Hanada, M. (1996) J Cell Biochem 60, 23-32; Amundson, S. A., Myers, T. G., Scudiero, D., Shinichi, K., Reed, J. C., and Fornace jr., A. J. (2000) Cancer Res 60, 6101-6110)

One possible approach is to identify compounds that mimic the BH3 domain of pro-apoptotic proteins and use them to disrupt the binding of BH3-containing proteins to anti-apoptotic Bcl-2 proteins, thus enabling the free BH3-containing proteins to initiate intrinsic apoptosis. The first study supporting this concept used a constrained BH3 peptide to induce apoptosis in cancer cells and to retard the growth of transplanted leukemia. (Wang, J. L., Zhang, Z. J., Choksi, S., Shan, S. M., Lu, Z. X., Croce, C. M., Alnemri, E. S., Korngold, R., and Huang, Z. W. (2000) Cancer Res 60, 1498-1502) Since then, several non-peptidic small molecule inhibitors have been identified. (Pellecchia, M., and Reed, J. C. (2004) Curr Pharm Des 10, 1387-1398; Mohammad, R. M., Goustin, A. S., Aboukameel, A., Chen, B., Banerjee, S., Wang, G., Nikolovska-Coleska, Z., Wang, S., and Al-Katib, A. (2007) Clin Cancer Res 13, 2226-2235; Paoluzzi, L., Gonen, M., Gardner, J. R., Mastrella, J., Yang, D., Holmlund, J., Sørensen, M., Leopold, L., Manova, K., Marcucci, G., Heaney, M. L., and O'Connor, O. A. (2008) Blood 111, 5350-5358)

To date, the most extensively studied and promising small molecule BH3 mimetic is ABT-737, which occupies the BH3 binding groove of Bcl-2, Bcl-X_{L}, and Bcl-w with high affinity, but only binds weakly to Mcl-1 and Bfl-1. (Oltersdorf, T., Elmore, S. W., Shoemaker, A. R., Armstrong, R. C., Augeri, D. J., Belli, B. A., Bruncko, M., Deckwerth, T. L., Dinges, J., Hajduk, P. J., Joseph, M. K., Kitada, S., Korsmeyer, S. J., Kunzer, A. R., Letai, A., Li, C., Mitten, M. J., Nettesheim, D. G., Ng, S., Nimmer, P. M., O'Connor, J. M., Oleksijew, A., Petros, A. M., Reed, J. C., Shen, W., Tahir, S. K., Thompson, C. B., Tomaselli, K. J., Wang, B., Wendt, M. D., Zhang, H., Fesik, S. W., and Rosenberg, S. H. (2005) Nature 435, 677-681; Konopleva, M., Contractor, R., Tsao, T., Samudio, I., Ruvolo, P. P., Kitada, S., Deng, X., Zhai, D., Shi, Y. X., Sneed, T., Verhaegen, M., Soengas, M., Ruvolo, V. R., McQueen, T., Schober, W. D., Watt, J. C., Jiffar, T., Ling, X., Marini, F. C., Harris, D., Dietrich, M., Estrov, Z., McCubrey, J., May, W. S., Reed, J. C., and Andreeff, M. (2006) Cancer Cell 10, 375-388)

Although much progress has been made over the last decade, further investigation is required to generate inhibitors targeting a broad class of anti-apoptotic Bcl-2 proteins. (Azmi, A. S., and Mohammad, R. M. (2009) J Cell Physiol 218, 13-21) This is important as both anti-apoptotic family sub-classes, Bcl-2/Bcl-X_{L}/Bcl-w and Mcl-1/Bfl-1, must be neutralized for apoptosis to occur. (Adams, J. M., and Cory, S. (2007) Oncogene 26, 1324-1337; Chen, L., Willis, S. N., Wei, A., Smith, B. J., Fletcher, J. I., Hinds, M. G., Colman, P. M., Day, C. L., Adams, J. M., and Huang, D. C. (2005) Mol Cell 17, 393-403; Willis, S. N., Chen, L., Dewson, G., Wei, A., Naik, E., Fletcher, J. I., Adams, J. M., and Huang, D. C. (2005) Genes Dev 19, 1294-1305)

The inventors have discovered a “pan-Bcl-2” inhibitor, BH3-M6, which is a synthetic terphenyl scaffold with functional groups that mimic the nature and the spatial configuration of the key amino acids in the BH3 α-helix. The inventors have found that BH3-M6 disrupts Bcl-2, Bcl-X_{L} and Mcl-1 binding to Bax, Bak, Bad or Bim, freeing up pro-apoptotic proteins, which leads to the release of cytochrome c, activation of caspases and induction of apoptosis in a Bax- and Bim-dependent manner in human cancer cells. The inventors have also developed a micelle comprising a multiblock copolymer having the pan-Bcl-2 inhibitor BH3-M6 encapsulated therein to increase bioavailability of the compound in the body.

### SUMMARY OF THE INVENTION

A critical hallmark of cancer cell survival is evasion of apoptosis. This is commonly due to overexpression of anti-apoptotic proteins such as Bcl-2, Bcl-X_{L} and Mcl-1, which bind to the BH3 α-helical domain of pro-apoptotic proteins such as Bax, Bak, Bad and Bim, and inhibit their function.

The inventors have designed a BH3 α-helical mimetic BH3-M6 that binds to Bcl-X_{L} and Mcl-1 and prevents their binding to fluorescently labeled Bak- or Bim-BH3 peptides in vitro. Using several approaches, the inventors demonstrate that BH3-M6 is a pan-Bcl-2 antagonist that inhibits the binding of Bcl-X_{L}, Bcl-2 and Mcl-1 to multi-domain Bax or Bak, or BH3-only Bim or Bad in cell-free systems and in intact human cancer cells, freeing up pro-apoptotic proteins to induce apoptosis. BH3-M6 disruption of these protein-protein interactions is associated with cytochrome c release from mitochondria, caspase-3 activation and PARP cleavage. Using caspase inhibitors and Bax and Bak siRNAs, the inventors demonstrate that BH3-M6-induced apoptosis is caspase- and Bax-, but not Bak-dependent. Furthermore, BH3-M6 disrupts Bcl-X_{L}/Bim, Bcl-2/Bim and Mcl-1/Bim protein-protein interactions and frees up Bim to induce apoptosis in human cancer cells that depend for tumor survival on the neutralization of Bim with Bcl-X_{L}, Bcl-2 or Mcl-1. BH3-M6 was also found to sensitize cells to apoptosis induced by the proteasome inhibitor CEP-1612. To increase solubility and bioavailability to the body for use as a therapeutic, the inventors have developed a micelle comprising a multiblock copolymer within which the pan-Bcl-2 inhibitor BH3-M6 is encapsulated.

The multiblock copolymer comprises a hydrophilic poly(ethylene glycol) block, a carboxylic acid-containing poly(amino acid) block, and a hydrophobic D,L-mixed poly(amino acid) block characterized in that the resulting micelle has an inner core, a carboxylic acid-containing outer core, and a hydrophilic shell. It will be appreciated that the hydrophilic poly(ethylene glycol) block corresponds to the hydrophilic shell, stabilizing carboxylic acid-containing poly(amino acid) block corresponds to the carboxylic acid-containing outer core, and the hydrophobic D,L-mixed poly(amino acid) block corresponds to the inner core.

In an embodiment, a compound comprised of the following chemical structure is presented:

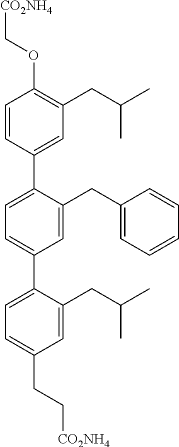

or pharmaceutically acceptable salts, prodrugs, salts of a prodrugs and metabolites thereof.

In another embodiments, a micelle is presented comprising a multiblock copolymer of formula I:

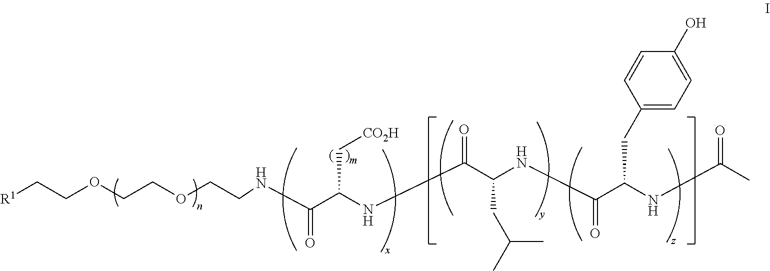

wherein:
   R¹ is —OCH₃, —N₃, or

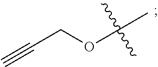 [SMILES: *OCC#C]

n is 110 to 450;
m is 1 or 2;
x is 3 to 50;
y is 5 to 50; and
z is 5 to 50;
wherein the micelle encapsulates BH3-M6 or pharmaceutically acceptable salts, prodrugs, salts of a prodrugs and metabolites thereof.

In another embodiment, a micelle is presented comprising a crosslinked multiblock polymer of formula III:

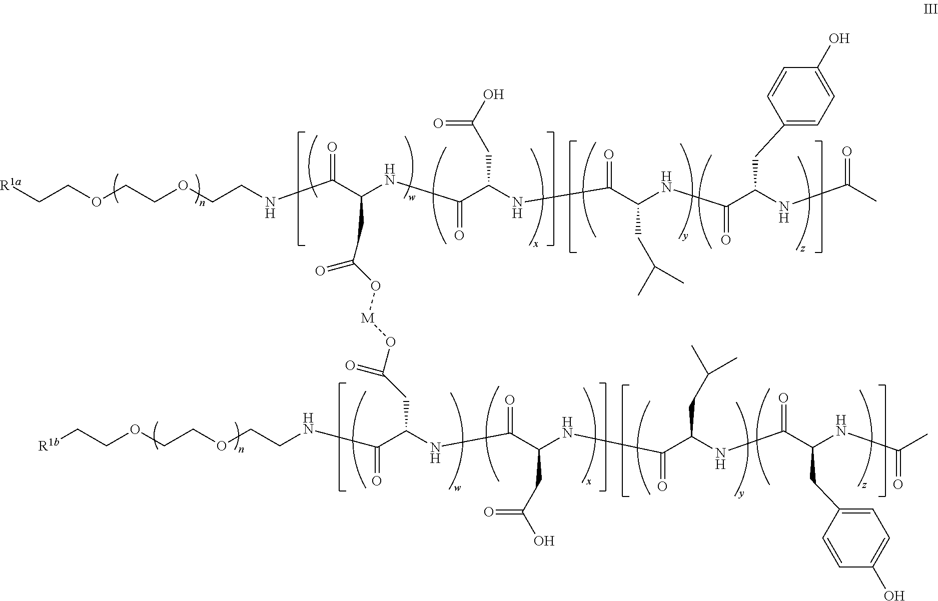

wherein:
   R^{1a} and R^{1b} are independently selected from —OCH₃, —N₃,

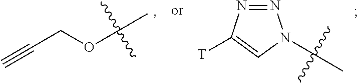

T is a targeting group moiety;
M is a metal ion;
n is 110 to 450;
w is 3 to 50;
x is 0 to 50, provided that the sum of w and x is no more than 50;
y is 5 to 50; and
z is 5 to 50;
wherein the micelle encapsulates BH3-M6 or pharmaceutically acceptable salts, prodrugs, salts of a prodrugs and metabolites thereof.

R^{1a} and R^{1b} may both be —CH₃ and M may be iron.

In another embodiment, a method of treating cancer is presented comprising administering a therapeutically effective amount of at least one pan-Bcl-2 inhibitor to a subject in need thereof wherein the at least one pan-Bcl-2 inhibitor is BH3-M6 or pharmaceutically acceptable salts, prodrugs, salts of a prodrugs and metabolites thereof.

The pan-Bcl-2 inhibitor may be encapsulated in a multiblock copolymer of Formula I or in a crosslinked multiblock polymer of Formula III. The cancer may be characterized by the overexpression of an anti-apoptotic Bcl-2 family protein. The cancer may be selected from the group consisting of breast cancer, non-small cell lung cancer, prostate cancer and adenocarcinoma.

At least one active agent may be administered with the at least one pan-Bcl-2 inhibitor. The active agent may be a proteasome inhibitor. The proteasome inhibitor may be selected from the group consisting of CEP-1612, bortezomib, NPI-0052, PR-171 and MG-132.

In another embodiment, a method of inducing apoptosis in a cancer cell is presented comprising administering at least one pan-Bcl-2 inhibitor to the cell wherein the at least one pan-Bcl-2 inhibitor is BH3-M6 or pharmaceutically acceptable salts, prodrugs, salts of a prodrugs and metabolites thereof. The pan-Bcl-2 inhibitor may be encapsulated in a multiblock copolymer of Formula I or in a crosslinked multiblock polymer of Formula III.

At least one active agent may be administered with the at least one pan-Bcl-2 inhibitor. The active agent may be a proteasome inhibitor. The proteasome inhibitor may be selected from the group consisting of CEP-1612, bortezomib, NPI-0052, PR-171 and MG-132.

In a further embodiment, a composition for treating cancer is presented comprising: at least one pan-Bcl-2 inhibitor wherein the at least one pan-Bcl-2 inhibitor is BH3-M6 or pharmaceutically acceptable salts, prodrugs, salts of a prodrugs and metabolites thereof; and a pharmaceutically acceptable carrier.

The pharmaceutically acceptable carrier may be a micelle multiblock copolymer of Formula I in which the at least one pan-Bcl-2 inhibitor is encapsulated in the micelle multiblock copolymer of Formula I.

The pharmaceutically acceptable carrier may be a micelle crosslinked multiblock polymer of Formula III in which the at least one pan-Bcl-2 inhibitor is encapsulated in the micelle crosslinked multiblock polymer of Formula III.

The cancer may be characterized by the overexpression of an anti-apoptotic Bcl-2 family protein. The cancer may be selected from the group consisting of breast cancer, non-small cell lung cancer, prostate cancer and adenocarcinoma.

At least one active agent may be administered with the at least one pan-Bcl-2 inhibitor. The active agent may be a proteasome inhibitor. The proteasome inhibitor may be selected from the group consisting of CEP-1612, bortezomib, NPI-0052, PR-171 and MG-132.

### BRIEF DESCRIPTION OF THE DRAWINGS

For a fuller understanding of the invention, reference should be made to the following detailed description, taken in connection with the accompanying drawings, in which:

FIG. 1 is a series of images illustrating the molecular docking studies of the interactions between BH3-M6 and Mcl-1, Bcl-X_{L}, and Bcl-2. (A) Chemical Structures of BH3-M6 and TPC. B-D, BH3-M6 docked to Mcl-1, Bcl-X_{L}, and Bcl-2. (B) The crystal structure of human Bim BH3 helix bound to human Mcl-1 with the side chains of Bim E55, 158, L62, 165, D67, and F69, relative to the BH3-M6 position. The protein Mcl-1 is shown by its molecular surface. Area with positive electrostatic potential is dark grey and that with negative electrostatic potential is light grey (the same presentation is used for proteins Bcl-X_{L} and Bcl2). (C) The residues of Mcl-1 interacting with BH3-M6. Dotted lines indicate hydrogen bonds. (D) The crystal structure of mouse Bim BH3 helix bound to mouse Bcl-X_{L} with the side chains of Bim I90, L94, I97, and D99, relative to the BH3-M6 position. (E) The residues of Bcl-X_{L} interacting with BH3-M6. Dotted lines indicate hydrogen bonds. (F) BH3-M6 docked to the original NMR structure of Bcl-2. (G) The residues of Bcl-2 that interact with BH3-M6.

The alignment between human and mouse Bim BH3 α-helix is as follows:

Human
(SEQ ID NO. 1)
(58-I A Q E L R R I G D E F N A Y-72)
Mouse
(SEQ ID NO. 2)
(90-I A Q E L R R I G D E F N E T-104)

FIG. 2 is a series of images illustrating that BH3-M6, but not TPC, inhibits the binding of anti-apoptotic proteins to pro-apoptotic proteins in vitro. (A) a series of graphs illustrating that BH3-M6 blocks the binding of Bak BH3 α-helix to Bcl-X_{L} (left) or Bim BH3 α-helix to Mcl-1 (right) as measured by fluorescence polarization assay, black circles: BH3-M6, open triangles: TPC. (B) BH3-M6 inhibits the interaction of Bcl-X_{L} or Mcl-1 with Bax and Bim as measured by GST pull-down assay. Purified GST, GST-Bcl-X_{L} and GST-Mcl-1 fusion proteins immobilized on glutathione-Sepharose beads were incubated for 1 h with 0, 50 and 100 μM BH3-M6 or 100 μM TPC, followed by addition of cell extracts from HEK293T or A549 cells for 1 h. Proteins associated with beads were eluted and analyzed by Western blotting with Bax and Bim antibodies. The inventors used GST antibodies either for negative controls (in lysates incubated with GST beads) or to demonstrate equal input (in lysates incubated with GST-Bcl-X_{L} or GST-Mcl-1 beads).

FIG. 3 is a series of images illustrating BH3-M6 inhibits the Bcl-X_{L} interaction with Bad in intact cells. BH3-M6 inhibits Bcl-X_{L}-induced GFP-Bad mitochondrial localization. COS-7 cells were grown in DMEM supplemented with 10% FBS and antibiotics, transfected with the indicated constructs, treated and analyzed as described in EXPERIMENTAL PROCEDURES. (A) cells expressing pEGFP-Bad and pcDNA3-HA-Bcl-X_{L}. (B) quantification of the fluorescence data (gray: diffuse, black: punctate cells). At least 85 transfected cells per sample were counted in three different fields of view.

FIG. 4 is a series of images illustrating BH3-M6 inhibits the interaction between Bcl-X_{L}, Bcl-2, and Mcl-1 with Bim, Bak or Bax in intact cells. (A) Co-immunoprecipitation from HEK293T cells. HEK293T cells were co-transfected with HA-Bcl-X_{L} and Flag-Bim_{EL} for 18 h. Cells were exposed to 0, 25, 50, and 100 μM of BH3-M6 or 100 μM of TPC for 2 h at 37° C., lysed and subjected to immunoprecipitation with anti-FLAG-M2 beads. The resulting immune complexes, as well as total lysates, were analyzed by Western blotting with the indicated antibodies. (B) Co-immunoprecipitation from MDA-MB-468 cells expressing Bcl-XL-IRES-Bim_{EL}, Bcl-2-IRES-Bim_{EL}, and Mcl-1-IRES-Bim_{EL}. Cells were grown in 100-mm plates and treated with 0, 25, 50, and 100 μM BH3-M6 or 100 μM TPC for 24 h at 37° C., lysed and subjected to immunoprecipitation with Bcl-X_{L}, Bcl-2, and Mcl-1 antibodies. The resulting immune complexes were analyzed by Western blotting with Bim, Bcl-X_{L}, Bcl-2, and Mcl-1 antibodies. (C) Co-immunoprecipitation from H1299 cells. H1299 non-small lung cancer cells were grown in RPMI-1640 medium plus 10% FBS, antibiotics and 1% sodium pyruvate, 1% HEPES, and 1.1% glucose. They were seeded in 100-mm plates and treated with 0, 25, 50, and 100 μM BH3-M6 or 100 μM TPC for 24 h at 37° C., lysed and subjected to immunoprecipitation with Bak antibody. The resulting immune complexes were analyzed by Western blotting with the indicated antibodies. (D) Co-immunoprecipitation from A549 cells. A549 cells were grown in F-12K medium plus 10% FBS and antibiotics and then serum-starved for 20 h, followed by treatment with 0, 25, and 50 μM of BH3-M6 or 50 μM of TPC for 1 h at 37° C. Cells were then lysed and subjected to immunoprecipitation with Bcl-X_{L} antibody (upper panel) and Bax 6A7 antibody (lower panel). The resulting immune complexes were analyzed by Western blotting with the antibodies indicated on the right.

FIG. 5 is a series of images illustrating BH3-M6 induces apoptosis in a caspase-dependent manner. (A) BH3-M6 releases cytochrome c from mitochondria. Mitochondria from A549 cells were incubated at 37° C. with 0, 50 μM BH3-M6 or TPC for 60 min. Cytochrome c release was determined as described in Methods. B-D A549 cells were treated with the indicated BH3-M6 concentrations for up to 72 h and then processed for various assays as described in EXPERIMENTAL PROCEDURES. (B) BH3-M6 induces apoptosis after 48 h of exposure to BH3-M6, as shown by phase images of cells (left panel) or DAPI nuclear staining (right panel). The arrows point towards cells in the process of rounding. The images from phase and DAPI are from different experiments. The graph below the images shows the percent of apoptotic cells and is the quantification of DAPI nuclear staining Error bars indicate standard deviations of triplicates. (C) BH3-M6 induces PARP cleavage after 48 h of drug treatment. (D) BH3-M6 decreases the viability of A549 cells in a dose-dependent manner as measured by MTT assay after 72 h treatment. Error bars indicate standard deviations of triplicates. (E) upper panel, Activation of caspase-3/-7 by BH3-M6 is blocked by a pan-caspase inhibitor. DoHH2 cells were pretreated with 50 μM Boc-D-FMK, a pan-caspase inhibitor, followed by a co-treatment with 50 μM BH3-M6 for 24 h. Caspase-3/-7 activity was determined as described in EXPERIMENTAL PROCEDURES. Results shown represent one of two independent experiments in triplicate. Error bars indicate standard deviations of triplicates. Middle panel, PARP cleavage was measured by Western blotting. Lower panel, extent of apoptosis was measured by TUNEL assay.

FIG. 6 is a series of images illustrating BH3-M6 induces apoptosis in a Bcl-2-, Bcl-X_{L}-, and Mcl-1-dependent manner. BH3-M6 induces caspase-3/-7 activities and apoptosis in Bcl-X_{L}/Bim, Bcl-2/Bim and Mcl-1/Bim overexpressing cells. MDA-MB-468 cells transfected with Bcl-X_{L}-IRES-Bim_{EL}, Bcl-2-IRES-Bim_{EL}, and Mcl-1-IRES-Bim_{EL} were grown in 100-mm plates and treated with 0, 25, 50, or 100 μM BH3-M6 or 100 μM of TPC for 24 h at 37° C. Cells were then harvested and lysed using 0.2% NP-40 lysis buffer (without protease and phosphatase inhibitors for cell-free caspase assay and with protease and phosphatase inhibitors for Western blot analysis). (A) Caspase-3/-7 activity was determined by incubating whole-cell extracts with caspase-3/-7 substrate and measuring free AMCs (left panel), apoptosis was quantified by TUNEL assay (middle panel), gray: TPC, black: BH3-M6. Bim_{EL}, Bcl-X_{L}, Bcl-2 and Mcl-1 expression levels were determined by Western blotting (right panel). (B) PARP cleavage by Western blotting. (C) Expression levels of different anti- and pro-apoptotic proteins in DU-145 and LNCaP cells as determined by Western blotting. (D) BH3-M6 induces apoptosis in LNCaP human prostate cancer cells expressing Bax, but not in DU-145 human prostate cancer cells lacking detectable Bax expression. LNCaP and DU-145 cells were treated with 0, 25 and 50 μM of BH3-M6 or 50 μM of TPC for 24 h. Cells were lysed and subjected to Western blot analysis. (E) Depletion of Bax, but not Bak by siRNA renders LNCaP cells resistant to apoptosis. LNCaP cells were transfected with 10 nM Bax or Bak siRNA or control siRNA for 48 h, followed by 24 h treatment with or without 25 or 50 μM BH3-M6. Cells were lysed and subjected to Western blot analysis with the indicated antibodies.

FIG. 7 is a series of images illustrating BH3-M6 sensitizes A549 cells to the proteasome inhibitor CEP-1612 to induce apoptosis. A549 cells were treated with the indicated concentrations of either drug alone or in combination and then processed for various assays as described in EXPERIMENTAL PROCEDURES. (A) Cell viability as measured by MTT assay after 72 h of treatment, error bars indicate standard deviations of triplicates. (B) apoptosis as determined by TUNEL assay after 48 h of treatment. (C) PARP cleavage after 24 hours of drug treatment.

FIG. 8 is an image depicting the dose-dependent cytotoxicity of A549 cancer cells for the free BH3M6 compound compared to the micelle formulation.

FIG. 9 is an image depicting the plasma pharmacokinetics of the BH3M6 compound at 30 mg/kg from free drug delivered i.p., or micelle formulation delivered i.v. or oral gavage.

FIG. 10 is an image depicting tumor accumulation of the BH3M6 compound at 30 mg/kg from free drug delivered i.p., or micelle formulation delivered i.v. or oral gavage.

### DETAILED DESCRIPTION OF THE PREFERRED EMBODIMENT

In the following detailed description of the preferred embodiments, reference is made to the accompanying drawings, which form a part hereof, and within which are shown by way of illustration specific embodiments by which the invention may be practiced. It is to be understood that other embodiments by which the invention may be practiced. It is to be understood that other embodiments may be utilized and structural changes may be made without departing from the scope of the invention.

### DEFINITIONS

As used in the specification and claims, the singular forms “a”, “an” and “the” include plural references unless the context clearly dictates otherwise.

All numerical designations, such as pH, temperature, time, concentration, and molecular weight, including ranges, are approximations which are varied up or down by increments of 1.0 or 0.1, as appropriate. It is to be understood, even if it is not always explicitly stated that all numerical designations are preceded by the term “about”. It is also to be understood, even if it is not always explicitly stated, that the reagents described herein are merely exemplary and that equivalents of such are known in the art and can be substituted for the reagents explicitly stated herein.

The term “about” or “approximately” as used herein refers to being within an acceptable error range for the particular value as determined by one of ordinary skill in the art, which will depend in part on how the value is measured or determined, i.e. the limitations of the measurement system, i.e. the degree of precision required for a particular purpose, such as a pharmaceutical formulation. For example, “about” can mean within 1 or more than 1 standard deviation, per the practice in the art. Alternatively, “about” can mean a range of up to 20%, preferably up to 10%, more preferably up to 5% and more preferably still up to 1% of a given value. Alternatively, particularly with respect to biological systems or processes, the term can mean within an order of magnitude, preferably within 5-fold, and more preferably within 2-fold, of a value. Where particular values are described in the application and claims, unless otherwise stated, the term “about” meaning within an acceptable error range for the particular value should be assumed.

The term “subject” as used herein describes an animal, preferably a human, to whom treatment is administered.

“Treatment” or “treating” as used herein refers to any of: the alleviation, amelioration, elimination and/or stabilization of a symptom or characteristic, as well as delay in progression of a symptom of a particular disorder. For example, “treatment” of cancer may include any one or more of the following: amelioration and/or elimination of one or more symptoms/characteristics associated with cancer, reduction of one or more symptoms/characteristics of cancer, stabilization of symptoms/characteristics of cancer, and delay in progression of one or more symptoms/characteristics of cancer.

As used herein, the term “therapeutically effective amount” is determined based on such considerations as known in the art including the recipient of the treatment, the recipient's tolerance for the compound, the disorder being treated, the severity of the disorder being treated, the composition containing the compound, the time of administration, the route of administration, the duration of treatment, the potency of the compound, the bioavailability of the compound, the rate of clearance of the compound from the body, and whether or not another active agent is co-administered. The amount of the compound of the instant invention that may be administered to a subject must be effective to achieve a response, including but not limited to, improved survival rate, more rapid recovery, and improvement or elimination of symptoms associated with cancers. In accordance with the present invention, a suitable single dose size is a dose that is capable of preventing or alleviating (reducing or eliminating) a symptom in a patient when administered one or more times over a suitable time period. One of ordinary skill in the art can readily determine appropriate single dose sizes for systemic administration based on the size of a mammal and the route of administration.

As used herein, “administering” or “administration” refers to the process by which the compounds of the present invention are delivered to a subject. The compounds of the present invention may be administered in a variety of ways including, but not limited to, bucally, opthalmically, orally, osmotically, parenterally (intramuscularly, intraperitoneally, intrasternally, intravenously, subcutaneously), rectally, topically, transdermally, or vaginally. Any of the compounds disclosed herein can be delivered to a subject by being covalently bound to albumin as part of a nanoparticle albumin bound delivery technology. Any of the compounds may also be delivered through encapsulation in vesicles such as liposomes, niosomes, micelles, etc.

The term “compound” as used herein refers to a chemical formulation, either organic or inorganic, that induces a desired pharmacological and/or physiological effect on a subject when administered in a therapeutically effective amount. “Compound” is used interchangeably herein with “drug” and “therapeutic agent”.

Compounds of the present invention may exist as acid addition salts, basic addition salts, zwitterions, prodrugs, salts of prodrugs, or metabolites. Salts of the compounds may be prepared during isolation or following purification of the compounds. Acid addition salts of the compounds are those derived from the reaction of the compounds with an acid such as, but not limited to, acetate; adipate; alginate; bicarbonate; citrate; aspartate; benzoate; benzenesulfonate; bisulfate; butyrate; camphorate; camphorsulfonate; digluconate; formate; fumarate; glycerophosphate; plutamate; hemisulfate; heptanoate; hexanoate; hydrochloride; hydrobromide; hydroiodide; lactobionate; lactate; maleate; mesitylenesulfonate; methanesulfonate; napthylenesulfonate; nicotinate; oxalate; pamoate; pectinate; persulfate; phosphate; picrate; propionate; succinate; tartrate; thiocynate; trichloroacetic, trifluoroacetic; para-toluenesulfonate; and undecanoate salts. Basic addition salts of the compounds are those derived from the reaction of the compounds with the hydroxide, carbonate, or bicarbonate cations such as lithium, sodium, potassium, calcium, and magnesium.

As used herein the term “pan-Bcl-2 inhibitor” refers to a compound that is capable of inhibiting both Bcl-2 anti-apoptotic family subclasses, Bcl-2/Bcl-X_{L}/Bcl-w and Mcl-1/Bfl-1 thus inducing apoptosis. BH3-M6 is an example of a pan-Bcl-2 inhibitor which is comprised of a synthetic terphenyl scaffold with functional groups that mimic the nature and spatial configuration of the key amino acids in the BH3 α-helix. Salts, prodrugs, salts of prodrugs and metabolites of BH3-M6 may also be considered pan-Bcl-2-inhibitors.

As used herein, an “active agent” refers to an agent or substance that has measurable specified or selected physiologic activity when administered to a subject in a therapeutically effective amount. Examples of active agents that are encompassed in the present invention include, but are not limited to, chemotherapeutics, proteasome inhibitors, apoptosis enhancers, apoptosis inducers, antitumor agents, alkylating agents, antimetabolites, anthracyclines, plant alkaloids, topoisomerase inhibitors, monoclonal antibodies, tyrosine kinase inhibitors, taxanes, anti-neoplastics, proteins, peptides, nucleic acid derivatives, polypeptides, and enzymes.

As used herein, a “proteasome inhibitor” refers to an active agent having anti-tumor activity in a cell, in vivo or in vitro, by disrupting the regulated degradation of pro-growth cell cycle proteins. This disruption may be through the induction of apoptosis. Examples of proteasome inhibitors include, but are not limited to, CEP-1612, bortezomib, ritonavir, NPI-0052, PR-171 and MG-132.

As used herein, the term “pharmaceutically acceptable carrier” is used to describe any of the standard pharmaceutically acceptable carriers. The pharmaceutically acceptable carrier can include excipients such as diluents, adjuvants, and vehicles, as well as implant carriers, and inert, non-toxic solid or liquid fillers, diluents, or encapsulating material that does not react with the active ingredients of the invention. Examples include, but are not limited to, phosphate buffered saline, physiological saline, water, and emulsions, such as oil/water emulsions. The carrier can be a solvent or dispersing medium containing, for example, ethanol, polyol (for example, glycerol, propylene glycol, liquid polyethylene glycol, and the like), suitable mixtures thereof, and vegetable oils. Formulations are described in a number of sources that are well known and readily available to those skilled in the art. For example, Remington's Pharmaceutical Sciences (Martin EW [1995] Easton Pa., Mack Publishing Company, 19^{th} ed.) describes formulations which can be used in connection with the subject invention.

Any of the compounds disclosed herein may be administered with or without an excipient. Excipients include, for example, encapsulating materials or additives such as absorption accelerators; antioxidants; binders; buffers; coating agents; coloring agents; diluents; disintegrating agents; emulsifiers; extenders; fillers; flavoring agents; humectants; lubricants; perfumes; preservatives; propellants; releasing agents; sterilizing agents; sweeteners; solubilizers; wetting agents; and mixtures thereof.

As used herein, “cancer” refers to any malignant neoplastic condition involving unregulated cell growth in which overexpression of Bcl-2 proteins is shown. Overexpression of Bcl-2 protein has previously been shown by those in the art to correlate with resistance to chemotherapy, clinical outcome, disease progression, overall prognosis or a combination thereof in several cancers. These cancers include, but are not limited to, hematologic and solid tumor types such as acoustic neuroma; acute leukemia; acute lymphoblastic leukemia; acute myelogenous leukemia (monocytic, myeoblastic, adenocarcinoma, angiosarcoma, astrocytoma, myelomonocytic, and promyelocytic); acute T-cell leukemia; basal cell carcinoma; bile duct carcinoma; bladder cancer; brain cancer; breast cancer (including estrogen-receptor positive breast cancer); bronchogenic carcinoma; Burkitt's lymphoma; cervical cancer; chondrosarcoma; chordoma; choriocarcinoma; chronic leukemia; chronic lymphocytic leukemia; chronic myelocytic (granulocytic) leukemia; chronic myelogenous leukemia; colon cancer; colorectal cancer; craniopharyngioma; cystadenocarcinoma; dysproliferative changes (dysplasias and metaplasias); embryonal carcinoma; endometrial cancer; endotheliosarcoma; ependymoma; epithelial carcinoma; erythroleukemia; esophageal cancer; essential thrombocythemia; Ewing's tumor; fibrosarcoma; gastric carcinoma; germ cell testicular cancer; gestational trophoblastic disease; glioblastoma; head and neck cancer; hemangioblastoma; hepatoma; hepatocellular cancer; hormone insensitive prostate cancer; leiomyosarcoma; liposarcoma; lung cancer (including non-small cell lung cancer and small cell lung cancer); lymphangioendothelio-sarcoma; lymphoangiosarcoma; lymphoblastic leukemia; lymphoma (including diffuse large B-cell lymphoma, follicular lymphoma, Hodgkin's lymphoma and non-Hodgkin's lymphoma); malignancies and hyperproliferative disorders of the bladder, breast, colon, lung, ovaries, pancreas, prostate, skin and uterus; lymphoid malignancies of T-cell or B-cell origin; leukemia; medullary carcinoma; medulloblastoma; melanoma; meningioma; mesothelioma; multiple myeloma; myelogenous leukemia; myeloma; myxosarcoma; neuroblastoma; oligodenroglioma; oral cancer; osteogenic sarcoma; ovarian cancer; pancreatic cancer; papillary adenocarcinomas; papillary carcinomas; peripheral T-cell lymphoma; pinealoma; polycythemia vera; prostate cancer (including hormone-insensitive (refractory) prostate cancer); rectal cancer; renal cell carcinoma; retinoblastoma; phabdomyosarcoma; sarcoma; sebaceous gland carcinoma; seminoma; skin cancer; small cell lung carcinoma; solid tumors (carcinomas and sarcomas); stomach cancer; squamous cell carcinoma; synovioma; sweat gland carcinoma; testicular cancer (including germ cell testicular cancer); thyroid cancer; Waldenstrom's macroglobulinemia; testicular tumors; uterine cancer; Wilm's tumor and the like.

As used herein, the term “multiblock copolymer” refers to a polymer comprising one synthetic polymer portion and two or more poly(amino acid) portions. Such multi-block copolymers include those having the format W—X′—X″, wherein W is a synthetic polymer portion and X and X′ are poly(amino acid) chains or “amino acid blocks”. In certain embodiments, the multiblock copolymers of the present invention are triblock copolymers. As described herein, one or more of the amino acid blocks may be “mixed blocks”, meaning that these blocks can contain a mixture of amino acid monomers thereby creating multiblock copolymers of the present invention. In some embodiments, the multiblock copolymers of the present invention comprise a mixed amino acid block and are tetrablock copolymers.

One skilled in the art will recognize that a monomer repeat unit is defined by parentheses depicted around the repeating monomer unit. The number (or letter representing a numerical range) on the lower right of the parentheses represents the number of monomer units that are present in the polymer chain. In the case where only one monomer represents the block (e.g. a homopolymer), the block will be denoted solely by the parentheses. In the case of a mixed block, multiple monomers comprise a single, continuous block. It will be understood that brackets will define a portion or block. For example, one block may consist of four individual monomers, each defined by their own individual set of parentheses and number of repeat units present. All four sets of parentheses will be enclosed by a set of brackets, denoting that all four of these monomers combine in random, or near random, order to comprise the mixed block. For clarity, the randomly mixed block of [BCADDCBADABCDABC] would be represented in shorthand by [(A)₄(B)₄(C)₄(D)₄].

As used herein, the term “triblock copolymer” refers to a polymer comprising one synthetic polymer portion and two poly(amino acid) portions.

As used herein, the term “inner core” as it applies to a micelle of the present invention refers to the center of the micelle formed by the hydrophobic D,L-mixed poly(amino acid) block. In accordance with the present invention, the inner core is not crosslinked. By way of illustration, in a triblock polymer of the format W—X′—X″, as described above, the inner core corresponds to the X″ block.

As used herein, the term “outer core” as it applies to a micelle of the present invention refers to the layer formed by the first poly(amino acid) block. The outer core lies between the inner core and the hydrophilic shell. In accordance with the present invention, the outer core is either crosslinkable or is crosslinked. By way of illustration, in a triblock polymer of the format W—X′—X″, as described above, the outer core corresponds to the X′ block. It is contemplated that the X′ block can be a mixed block.

As used herein, the terms “drug-loaded” and “encapsulated”, and derivatives thereof, are used interchangeably. In accordance with the present invention, a “drug-loaded” micelle refers to a micelle having a drug, compound or therapeutic agent, situated within the core of the micelle. In certain instances, the drug, compound or therapeutic agent is situated at the interface between the core and the hydrophilic corona. This is also referred to as a drug, compound or therapeutic agent, being “encapsulated” within the micelle.

As used herein, the term “polymeric hydrophilic block” refers to a polymer that is not a poly(amino acid) and is hydrophilic in nature. Such hydrophilic polymers are well known in the art and include polyethyleneoxide (also referred to as polyethylene glycol or PEG), and derivatives thereof, poly(N-vinyl-2-pyrolidone), and derivatives thereof, poly(N-isopropylacrylamide), and derivatives thereof, poly(hydroxyethyl acrylate), and derivatives thereof, poly(hydroxylethyl methacrylate), and derivatives thereof, and polymers of N-(2-hydroxypropoyl)methacrylamide (HMPA) and derivatives thereof.

As used herein, the term “poly(amino acid)” or “amino acid block” refers to a covalently linked amino acid chain wherein each monomer is an amino acid unit. Such amino acid units include natural and unnatural amino acids. In certain embodiments, each amino acid unit of the optionally a crosslinkable or crosslinked poly(amino acid block) is in the L-configuration. Such poly(amino acids) include those having suitably protected functional groups. For example, amino acid monomers may have hydroxyl or amino moieties which are optionally protected by a suitable hydroxyl protecting group or a suitable amine protecting group, as appropriate. Such suitable hydroxyl protecting groups and suitable amine protecting groups are described in more detail herein, infra. As used herein, an amino acid block comprises one or more monomers or a set of two or more monomers. In certain embodiments, an amino acid block comprises one or more monomers such that the overall block is hydrophilic. In still other embodiments, amino acid blocks of the present invention include random amino acid blocks, i.e. blocks comprising a mixture of amino acid residues.

As used herein, the term “D,L-mixed poly(amino acid) block” refers to a poly(amino acid) block wherein the poly(amino acid) consists of a mixture of amino acids in both the D- and L-configurations. In certain embodiments, the D,L-mixed poly(amino acid) block is hydrophobic. In other embodiments, the D,L-mixed poly(amino acid) block consists of a mixture of D-configured hydrophobic amino acids and L-configured hydrophilic amino acid side-chain groups such that the overall poly(amino acid) block comprising is hydrophobic.

Exemplary poly(amino acids) include poly(benzyl glutamate), poly(benzyl aspartate), poly(L-leucine-co-tyrosine), poly(D-leucine-co-tyrosine), poly(L-phenylalanine-co-tyrosine), poly(D-phenylalanine-co-tyrosine), poly(L-leucine-coaspartic acid), poly(D-leucine-co-aspartic acid), poly(L-phenylalanine-co-aspartic acid), poly(D-phenylalanine-co-aspartic acid).

As used herein, the phrase “natural amino acid side-chain group” refers to the side-chain group of any of the 20 amino acids naturally occurring in proteins. Such natural amino acids include the nonpolar, or hydrophobic amino acids, glycine, alanine, valine, leucine isoleucine, methionine, phenylalanine, tryptophan, and proline. Cysteine is sometimes classified as nonpolar or hydrophobic and other times as polar. Natural amino acids also include polar, or hydrophilic amino acids, such as tyrosine, serine, threonine, aspartic acid (also known as aspartate, when charged), glutamic acid (also known as glutamate, when charged), asparagine, and glutamine. Certain polar, or hydrophilic, amino acids have charged side-chains. Such charged amino acids include lysine, arginine, and histidine. One of ordinary skill in the art would recognize that protection of a polar or hydrophilic amino acid side-chain can render that amino acid nonpolar. For example, a suitably protected tyrosine hydroxyl group can render that tyrosine nonpolar and hydrophobic by virtue of protecting the hydroxyl group.

As used herein, the phrase “unnatural amino acid side-chain group” refers to amino acids not included in the list of 20 amino acids naturally occurring in proteins, as described above. Such amino acids include the D-isomer of any of the 20 naturally occurring amino acids. Unnatural amino acids also include homoserine, ornithine, and thyroxine. Other unnatural amino acids side-chains are well known to one of ordinary skill in the art and include unnatural aliphatic side chains. Other unnatural amino acids include modified amino acids, including those that are N-alkylated, cyclized, phosphorylated, acetylated, amidated, azidylated, labeled, and the like.

As used herein, the term “tacticity” refers to the stereochemistry of the poly(amino acid) hydrophobic block. A poly(amino acid) block consisting of a single stereoisomer (e.g. all L isomer) is referred to as “isotactic”. A poly(amino acid) consisting of a random incorporation of D and L amino acid monomers is referred to as an “atactic” polymer. A poly(amino acid) with alternating stereochemistry (e.g. . . . DLDLDL . . . ) is referred to as a “syndiotactic” polymer. Polymer tacticity is described in more detail in “Principles of Polymerization”, 3rd Ed., G. Odian, John Wiley & Sons, New York: 1991, the entire contents of which are hereby incorporated by reference.

The term “aliphatic” or “aliphatic group”, as used herein, denotes a hydrocarbon moiety that may be straight-chain (i.e., unbranched), branched, or cyclic (including fused, bridging, and spiro-fused polycyclic) and may be completely saturated or may contain one or more units of unsaturation, but which is not aromatic. Unless otherwise specified, aliphatic groups contain 1-20 carbon atoms. In some embodiments, aliphatic groups contain 1-10 carbon atoms. In other embodiments, aliphatic groups contain 1-8 carbon atoms. In still other embodiments, aliphatic groups contain 1-6 carbon atoms, and in yet other embodiments aliphatic groups contain 1-4 carbon atoms. Suitable aliphatic groups include, but are not limited to, linear or branched, alkyl, alkenyl, and alkynyl groups, and hybrids thereof such as (cycloalkyl)alkyl, (cycloalkenyl)alkyl or (cycloalkyl)alkenyl.

Unless otherwise stated, structures depicted herein are also meant to include all isomeric (e.g., enantiomeric, diastereomeric, and geometric (or conformational)) forms of the structure; for example, the R and S configurations for each asymmetric center, Z and E double bond isomers, and Z and E conformational isomers. Therefore, single stereochemical isomers as well as enantiomeric, diastereomeric, and geometric (or conformational) mixtures of the present compounds are within the scope of the invention. Unless otherwise stated, all tautomeric forms of the compounds of the invention are within the scope of the invention. Additionally, unless otherwise stated, structures depicted herein are also meant to include compounds that differ only in the presence of one or more isotopically enriched atoms. For example, compounds having the present structures except for the replacement of hydrogen by deuterium or tritium, or the replacement of a carbon by a ¹³C- or ¹⁴C-enriched carbon are within the scope of this invention. Such compounds are useful, for example, as in neutron scattering experiments, as analytical tools or probes in biological assays.

### BH3-M6 Compound

One of the major determinants of cell survival is the balance between the anti-apoptotic and pro-apoptotic members of the Bcl-2 family, and overexpression of anti-apoptotic proteins such as Bcl-2, Bcl-X_{L}, and Mcl-1 contributes to cancer progression and confers resistance to chemotherapy and radiation therapy. (Adams, J. M., and Cory, S. (2007) Oncogene 26, 1324-1337; Reed, J. C., Miyashita, T., Takayama, S., Wang, H. G., Sato, T., Krajewski, S., Aime-Sempe, C., Bodrug, S., Kitada, S., and Hanada, M. (1996) J Cell Biochem 60, 23-32; Amundson, S. A., Myers, T. G., Scudiero, D., Shinichi, K., Reed, J. C., and Formace jr., A. J. (2000) Cancer Res 60, 6101-6110) For example, Bcl-2 overexpression is a key molecular feature of drug resistance of non-Hodgkin's lymphoma patients to chemotherapy, and high Mcl-1 levels in chronic lymphocytic leukemia patients correlate with a decreased complete response to chemotherapy. (Mohammad, R. M., Goustin, A. S., Aboukameel, A., Chen, B., Banerjee, S., Wang, G., Nikolovska-Coleska, Z., Wang, S., and Al-Katib, A. (2007) Clin Cancer Res 13, 2226-2235; Kramer, M. H., Hermans, J., Wijburg, E., Philippo, K., Geelen, E., van Krieken, J. H., de Jong, D., Maartense, E., Schuuring, E., and Kluin, P. M. (1998) Blood 92, 3152-3162; Iqbal, J., Neppalli, V. T., Wright, G., Dave, B. J., Horsman, D. E., Rosenwald, A., Lynch, J., Hans, C. P., Weisenburger, D. D., Greiner, T. C., Gascoyne, R. D., Campo, E., Ott, G., Muller-Hermelink, H. K., Delabie, J., Jaffe, E. S., Grogan, T. M., Connors, J. M., Vose, J. M., Armitage, J. O., Staudt, L. M., and Chan, W. C. (2006) J Clin Oncol 24, 961-968; Kitada, S., Andersen, J., Akar, S., Zapata, J. M., Takayama, S., Krajewski, S., Wang, H. G., Zhang, X., Bullrich, F., Croce, C. M., Rai, K., Hines, J., and Reed, J. C. (1998) Blood 91, 3379-3389; Saxena, A., Viswanathan, S., Moshynska, O., Tandon, P., Sankaran, K., and Sheridan, D. P. (2004) Am J Hematol 75, 22-33) Therefore, an important goal of cancer drug discovery is to target the anti-apoptotic pathways regulated by Bcl-2 family proteins.

The ability of anti-apoptotic proteins to preserve cancer cell survival depends on protein-protein interactions involving the binding of the amphipathic α-helical BH3 domain of pro-apoptotic proteins such as Bax, Bak, Bad and Bim to a hydrophobic pocket formed by the BH1, BH2, and BH3 domains at the surface of anti-apoptotic proteins. (Youle, R. J. (2007) Science 315, 776-777; Jiang, X., and Wang, X. (2004) Annu Rev Biochem 73, 87-106) This prompted a search for BH3 domain mimics as potential novel anti-cancer drugs. (Azmi, A. S., and Mohammad, R. M. (2009) J Cell Physiol 218, 13-21; Vogler, M., Dinsdale, D., Dyer, M. J., and Cohen, G. M. (2009) Cell Death Differ 16, 360-367) Determination of the structure of several anti-apoptotic proteins and the development of high-throughput screening assays led to the identification of many antagonists of anti-apoptotic proteins. However, except for a few such as ABT-737 and ABT-263, a thorough characterization of their specificity has not been carried out, and with some of these antagonists, off-target effects may contribute to their activity. (van Delft, M. F., Wei, A. H., Mason, K. D., Vandenberg, C. J., Chen, L., Czabotar, P. E., Willis, S, N., Scott, C. L., Day, C. L., Cory, S., Adams, J. M., Roberts, A. W., and Huang, D. C. (2006) Cancer Cell 10, 389-399) Furthermore, some Bcl-2 antagonists such as ABT-737 are highly specific for one sub-class of anti-apoptotic proteins (i.e. Bcl-2, Bcl-X_{L} and Bcl-w) but not the other sub-class (i.e. Mcl-1 and Bfl-1). (van Delft, M. F., Wei, A. H., Mason, K. D., Vandenberg, C. J., Chen, L., Czabotar, P. E., Willis, S. N., Scott, C. L., Day, C. L., Cory, S., Adams, J. M., Roberts, A. W., and Huang, D. C. (2006) Cancer Cell 10, 389-399) However, apoptosis induction requires antagonizing both sub-classes of anti-apoptotic proteins. (Adams, J. M., and Cory, S. (2007) Oncogene 26, 1324-1337; Chen, L., Willis, S. N., Wei, A., Smith, B. J., Fletcher, J. I., Hinds, M. G., Colman, P. M., Day, C. L., Adams, J. M., and Huang, D. C. (2005) Mol Cell 17, 393-403; Willis, S. N., Chen, L., Dewson, G., Wei, A., Naik, E., Fletcher, J. I., Adams, J. M., and Huang, D. C. (2005) Genes Dev 19, 1294-1305)

The inventors have developed an α-helix mimetic BH3-M6 that disrupts the interactions between Bcl-2, Bcl-X_{L}, and Mcl-1 with Bax, Bak, Bim or Bad and leads to cytochrome c release, caspase activation, PARP cleavage and apoptosis in human cancer cells. It induced Bax conformational change and required Bax expression and caspase activation to induce apoptosis.

While several antagonists of anti-apoptotic proteins such as Gossypol, Apogossypolone, TW-37, Obatoclax, ABT-737, ABT-263, HA1-41, Chelerythrine, Antimycin, BHI-1 and others have been identified, only a few have been thoroughly characterized for their specificity to disrupt protein-protein interactions between various class of pro- and anti-apoptotic proteins. (Azmi, A. S., and Mohammad, R. M. (2009) J Cell Physiol 218, 13-21; Vogler, M., Dinsdale, D., Dyer, M. J., and Cohen, G. M. (2009) Cell Death Differ 16, 360-367)

Unlike other antagonists of anti-apoptotic proteins, BH3-M6 was designed based on the α-helix mimicry strategy that uses a terphenyl scaffold to spatially project functionality in a manner similar to that of two turns of the BH3 domain α-helix. (Yin, H., Lee, G. I., Sedey, K. A., Kutzki, 0., Park, H. S., Orner, B. P., Ernst, J. T., Wang, H. G., Sebti, S. M., and Hamilton, A. D. (2005) J Am Chem Soc 127, 10191-10196) The computational docking studies suggest that BH3-M6 binds at the BH3 binding cleft of anti-apoptotic proteins and engages amino acid residues that are involved in binding to the BH3 α-helices of pro-apoptotic proteins. The unsubstituted control compound TPC makes few favorable contacts, confirming that the side chains attached to the terphenyl scaffold are required for interaction with Bcl-2, Bcl-X_{L} and Mcl-1. Consistent with the docking results, fluorescence polarization assays showed that BH3-M6 displaced the Bcl-X_{L}- and Mcl-1-bound Bak or Bim peptides, suggesting that BH3-M6 disrupts Bcl-X_{L}/Bak and Mcl-1/Bim interactions by binding the BH3 binding site on Bcl-X_{L} and Mcl-1.

Using several approaches, the inventors show that BH3-M6 disrupted Bcl-X_{L}, Bcl-2, and Mcl-1 interactions with Bax, Bak, Bad or Bim, suggesting that BH3-M6 is a broad pan-Bcl-2 antagonist capable of antagonizing the two distinct sub-classes of anti-apoptotic proteins, which is critical for full induction of apoptosis. (Adams, J. M., and Cory, S. (2007) Oncogene 26, 1324-1337; Chen, L., Willis, S. N., Wei, A., Smith, B. J., Fletcher, J. I., Hinds, M. G., Colman, P. M., Day, C. L., Adams, J. M., and Huang, D. C. (2005) Mol Cell 17, 393-403; Willis, S. N., Chen, L., Dewson, G., Wei, A., Naik, E., Fletcher, J. I., Adams, J. M., and Huang, D. C. (2005) Genes Dev 19, 1294-1305) Other published antagonists including ABT-737 and ABT-263 are not pan-Bcl-2 inhibitors and on their own may not be able to induce apoptosis in tumors where both Bcl-X_{L} and Mcl-1 are overexpressed. (Azmi, A. S., and Mohammad, R. M. (2009) J Cell Physiol 218, 13-21; Vogler, M., Dinsdale, D., Dyer, M. J., and Cohen, G. M. (2009) Cell Death Differ 16, 360-367) This is consistent with studies that showed that Mcl-1 confers resistance to Bcl-2-, Bcl-X_{L}- and Bcl-w-selective antagonists such as ABT-737, and that Obatoclax, which antagonizes both sub-classes, overcomes this resistance. (Nguyen, M., Marcellus, R. C., Roulston, A., Watson, M., Serfass, L., Murthy Madiraju, S. R., Goulet, D., Viallet, J., Belec, L., Billot, X., Acoca, S., Purisima, E., Wiegmans, A., Cluse, L., Johnstone, R. W., Beauparlant, P., and Shore, G. C. (2007) Proc Natl Acad Sci USA 104, 19512-19517) Since BH3-M6 disrupted the interaction of anti-apoptotic proteins with both multi-domain and BH3-only pro-apoptotic proteins, this compound represents an important advantage, since several mechanisms have been proposed for Bcl-2 family-mediated cancer cell survival including direct and indirect ones that involve neutralization by anti-apoptotic proteins of either multi-domain or BH3-only pro-apoptotic proteins. (Willis, S. N., and Adams, J. M. (2005) Curr Opin Cell Biol 17, 617-625; Ewings, K. E., Wiggins, C. M., and Cook, S. J. (2007) Cell Cycle 6, 2236-2240)

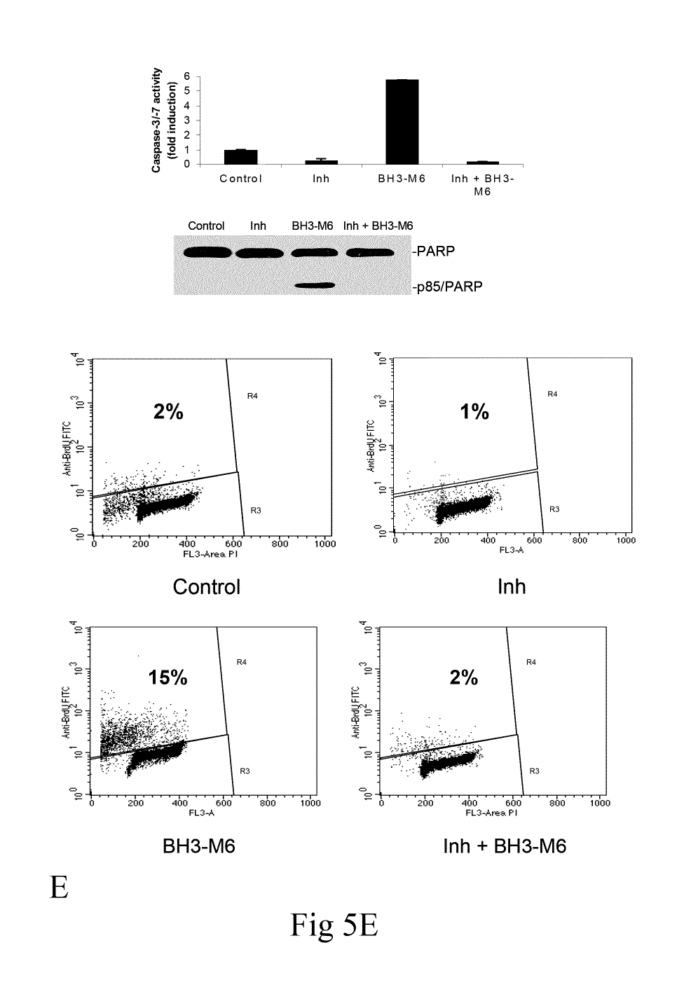

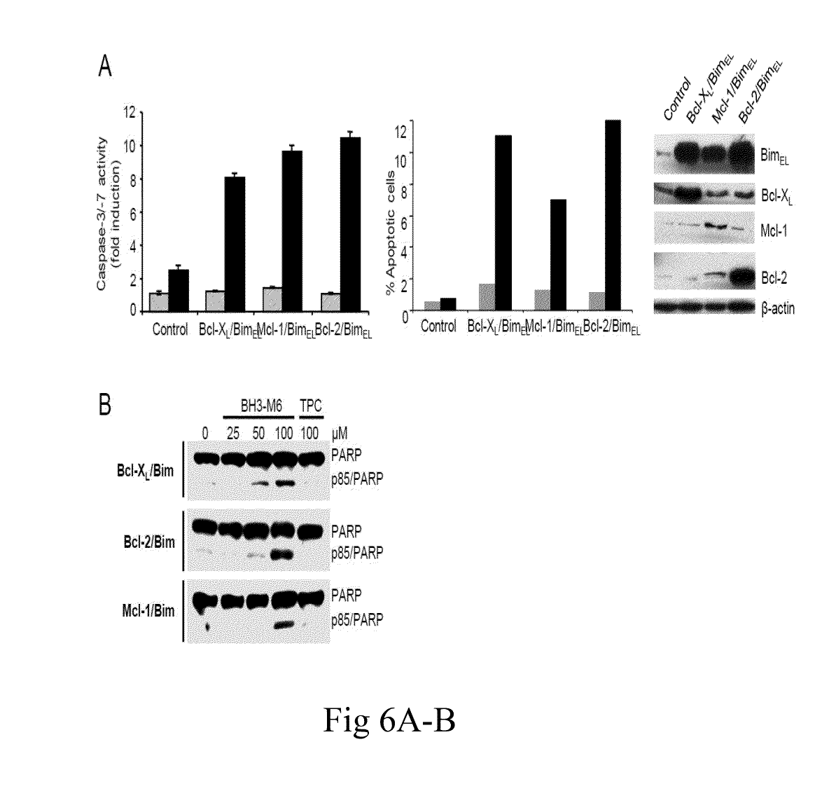

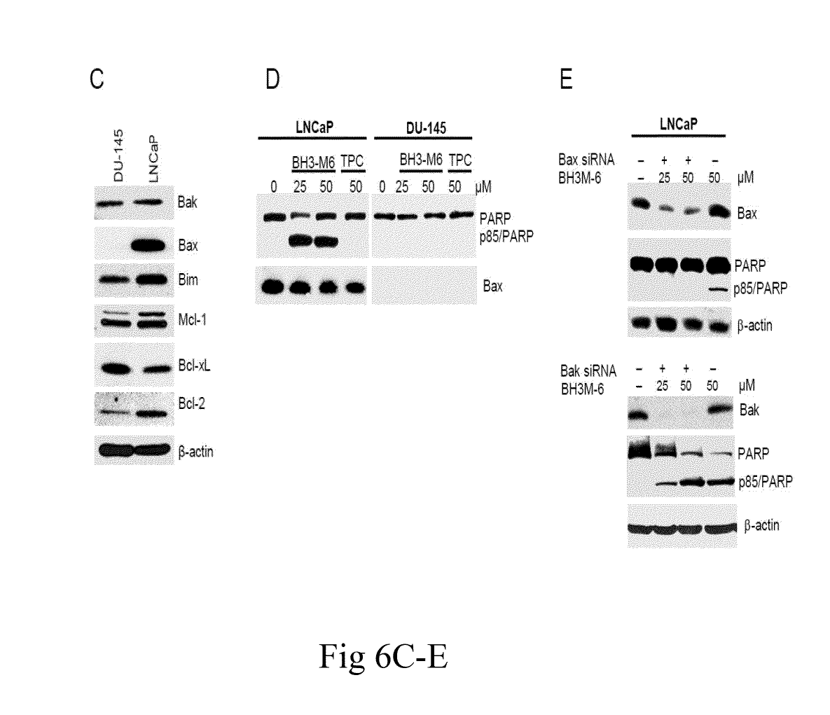

It is also important to note that some antagonists of anti-apoptotic proteins have other targets and may kill cells by mechanisms that are independent of activation of Bax and Bak. Indeed, Gossypol, Antimycin, BHI-1, HA1-41 and Chelerythrine kill wild-type and Bax/Bak-deficient fibroblasts with similar efficacy. (van Delft, M. F., Wei, A. H., Mason, K. D., Vandenberg, C. J., Chen, L., Czabotar, P. E., Willis, S. N., Scott, C. L., Day, C. L., Cory, S., Adams, J. M., Roberts, A. W., and Huang, D. C. (2006) Cancer Cell 10, 389-399) In contrast, the same study showed that ABT-737 kills only wild-type, but not Bax/Bak-deficient cells, suggesting that this compound is a true BH3 mimic that induces apoptosis by specifically binding anti-apoptotic proteins and releasing pro-apoptotic proteins. The data presented in FIGS. 6A-B suggest that in cells depending for survival on anti-apoptotic proteins neutralizing Bim, BH3-M6-induced apoptosis depends on its ability to disrupt the interaction of Bim with Bcl-X_{L}, Bcl-2, or Mcl-1. This frees up Bim allowing it to induce apoptosis, possibly by directly activating Bax and/or Bak as has been suggested previously. (Yamaguchi, H., and Wang, H. G. (2002) J Biol Chem 277, 41604-41612) Furthermore, the data presented in FIGS. 6C-E suggest that BH3-M6-induced apoptosis depends on Bax. These results suggest that BH3-M6 induces apoptosis by freeing up Bax from anti-apoptotic proteins and/or directly inducing Bax to assume its pro-apoptotic conformation.

The ability of BH3-M6 to inhibit the binding of Bcl-2, Bcl-X_{L}, and Mcl-1 to pro-apoptotic proteins is predicted to free up Bax, Bak, Bim and Bad. However, these pro-apoptotic proteins can be degraded by the proteasome. Therefore, a combination of BH3-M6 and a proteasome inhibitor are more effective than single agent treatment. The combination of BH3-M6 with the proteasome inhibitor CEP-1612 synergistically kills A549 cells. The results are consistent with others that show that ABT-737 synergizes with the proteasome inhibitors bortezomib or MG-132. (Paoluzzi, L., Gonen, M., Bhagat, G., Furman, R. R., Gardner, J. R., Scotto, L., Gueorguiev, V. D., Heaney, M. L., Manova, K., and O'Connor, O. A. (2008) Blood 112, 2906-2916; Miller, L. A., Goldstein, N. B., Johannes, W. U., Walton, C. H., Fujita, M., Norris, D. A., and Shellman, Y. G. (2009) J Invest Dermatol 129, 964-971)

### Experimental Procedures

### Antibodies

Antibodies to human proteins were from the following sources: cytochrome c (BD PharMingen, San Diego, Calif.); Cox IV and poly(ADP-ribose) polymerase (PARP) (Roche, Indianapolis, Ind.); GST, Bcl-X_{L}, Bax (N20), Bcl-2, Mcl-1 (Santa Cruz Biotechnology, Santa Cruz, Calif.); Bax (6A7), HA, FLAG-M2 (Sigma, St. Louis, Mo.); Bim (Epitomics, Burlingame, Calif.); Bak (Millipore, Temecula, Calif.).

### Molecular Modeling

Compound docking was carried out using the GLIDE (Grid Based Ligand Docking from Energetics) Program from Schrödinger, L. L. C. (Friesner, R. A., Banks, J. L., Murphy, R. B., Halgren, T. A., Klicic, J. J., Mainz, D. T., Repasky, M. P., Knoll, E. H., Shelley, M., Perry, J. K., Shaw, D. E., Francis, P., and Shenkin, P. S. (2004) J Med Chem 47, 1739-1749; Halgren, T. A., Murphy, R. B., Friesner, R. A., Beard, H. S., Frye, L. L., Pollard, W. T., and Banks, J. L. (2004) J Med Chem 47, 1750-1759) The Jorgensen OPLS-2001 force field was applied in the GLIDE program. The optimal binding geometry for each model was obtained by utilization of Monte Carlo sampling techniques coupled with energy minimization. GLIDE uses a scoring method based on ChemScore but with additional terms added for greater accuracy. GLIDE 4.5 SP (Standard Precision mode) was used to dock each chemical structure of these compounds followed by GLIDE 4.5 XP (Extra Precision mode) docking to find probable conformational hits. An X-ray crystal structure of mouse Bcl-X_{L} in complex with mouse Bim BH3 at 1.65 Å resolution (1PQ1.pdb) was used for Bcl-X_{L} docking, and an X-ray crystal structure of human Mcl-1 in complex with human Bim BH3 at 1.55 Å resolution (2NL9.pdb) was used for Mcl-1 docking (Liu, X., Dai, S., Zhu, Y., Marrack, P., and Kappler, J. W. (2003) Immunity 19, 341-352; Czabotar, P. E., Lee, E. F., van Delft, M. F., Day, C. L., Smith, B. J., Huang, D. C., Fairlie, W. D., Hinds, M. G., and Colman, P. M. (2007) Proc Natl Acad Sci USA 104, 6217-6222) An NMR solution structure of the human Bcl-2 in complex with an acyl-sulfonamide-based ligand (202F.pdb) was used for Bcl-2 docking (Bruncko, M., Oost, T. K., Belli, B. A., Ding, H., Joseph, M. K., Kunzer, A., Martineau, D., McClellan, W. J., Mitten, M., Ng, S. C., Nimmer, P. M., Oltersdorf, T., Park, C. M., Petros, A. M., Shoemaker, A. R., Song, X., Wang, X., Wendt, M. D., Zhang, H., Fesik, S. W., Rosenberg, S. H., and Elmore, S. W. (2007) J Med Chem 50, 641-662)

### Cell Culture

Human and simian cells were obtained from the American Type Culture Collection. All cell culture media were supplemented with 10% fetal calf serum, 100 units/ml penicillin, and 100 μg/ml streptomycin, and maintained at 37° C. and 5% CO₂. In addition, H1299 cells were supplemented with 1% sodium pyruvate, 1% HEPES, and 1.1% glucose.

### Co-Immunoprecipitation

HEK293T cells were co-transfected with 5 μg of pcDNA3, pCMV2, pcDNA3-HA-Bcl-X_{L} or pCMV2-Flag-Bim_{EL} expression vectors using TransFectin™ reagent (Bio-Rad Laboratories, Hercules, Calif.) and, after 18 h, exposed to DMSO, BH3-M6, or TPC (terphenyl control) for 2 h at 37° C. Cell lysates were prepared in NP-40 lysis buffer (10 mM HEPES pH 7.5, 142.5 mM KCl, 5 mM MgCl₂, 1 mM EGTA, 0.2% NP-40, 2 mM Na₃VO₄, 2 mM phenylmethylsulphonyl fluoride (PMSF), 6.4 mg/ml p-nitrophenylphosphate, and 1× Halt™ EDTA-free protease inhibitor cocktail (Pierce, Rockford, Ill.)). Bim was immunoprecipitated from cell lysates containing 100 μg protein with 2 μg FLAG-M2 beads (Sigma, St. Louis, Mo.) in 500 μl of the same lysis buffer at 4° C. overnight. Beads were washed four times with lysis buffer, boiled for 5 min in Laemmli sample buffer and analyzed by Western blotting.

A549 cells are a type of cell line that is derived from adenocarcinoma human alveolar basal epithelial cells. A549 cells were serum-starved for 20 h and treated with TPC or different concentrations of BH3-M6 for 1 h at 37° C. Whole cell lysates were prepared as described above and subjected to immunoprecipitation. In the case of A549 cells, 230 μg protein was incubated with 1.5 μg Bcl-X_{L} antibody in 250 μl NP-40 lysis buffer overnight at 4° C. Immunoprecipitates were collected by adding 25 μl protein A/G agarose beads (Santa Cruz Biotechnology) for 2 h at 4° C., followed by centrifugation for 2 min at 3000 g. The beads were processed as described above.

MDA-MB-468 cells, a breast cancer cell line, expressing Bcl-X_{L}-IRES-Bim, Bcl-2-IRES-Bim, Mcl-1-IRES-Bim and H1299 cells, a human non-small cell lung carcinoma cell line, were treated with TPC or different concentrations of BH3-M6 for 24 h at 37° C. Whole cell lysates were prepared as described above and subjected to immunoprecipitation by incubating 500 μg protein with 1.5 μg of Bcl-X_{L}, Bcl-2, Mcl-1 or 4 μg of Bak antibodies in 250 μA NP-40 lysis buffer overnight at 4° C. The remainder of the procedure was the same as above.

To detect conformationally changed Bax, 20 h serum-starved A549 cells were treated with TPC or different concentrations of BH3-M6 for 1 h, whole cell lysates were prepared in CHAPS lysis buffer containing protease inhibitors, and 200 μg protein was incubated with 2 μg Bax 6A7 antibody for 2 h at 4° C. Then 20 μl of protein G agarose (Millipore) was added into the reactions and incubated at 4° C. for an additional 2 h, followed by washing with the same lysis buffer, and the immunoprecipitates were immunoblotted with Bax N20 polyclonal antibody.

In Vitro Protein Protein Interactions (GST Pull-Down Assay)

Whole cell lysates of HEK293T and A549 cells were prepared using NP-40 lysis buffer (see above). For in vitro binding assay, 1 μg of GST, GST-Bcl-X_{L} or GST-Mcl-1 fusion proteins were coupled to 40 μl prewashed Glutathione Sepharose™ 4B beads (GE Healthcare, Piscataway, N.J.) in 250 μl of NP-40 lysis buffer with continuous rocking for 1 h at room temperature. The beads were centrifuged and washed four times with cold PBS and once with NP-40 lysis buffer. DMSO, TPC, or different concentrations of BH3-M6 in 100 μl NP-40 lysis buffer were added and incubated at 4° C. for 1 h with continuous rocking Whole cell lysates from HEK293T or A549 cells containing 500 μg of protein were added to beads and again incubated at 4° C. for 3 h with continuous rocking. The beads were then processed for Western blotting as described above.

### Fluorescence Microscopy Analysis

COS-7 cells were seeded onto glass cover slips in 24-well plates and transfected with 1 μg pEGFP-Bad expression vector alone or co-transfected with 0.5 μg pEGFP-Bad and 0.5 μg pcDNA3-HA-Bcl-X_{L} sing TransFectin™ reagent. After 18 h, cells were treated with DMSO, 100 μM of BH3-M6, or TPC for 4 h in the presence of a caspase inhibitor (50 μM z-VAD-fmk) to prevent apoptosis. After three washes with 500 μl cold PBS, cells were fixed with 3.7% paraformaldehyde for 5 min at room temperature. Cells were washed again three times with 500 μl cold PBS, then permeabilized with 0.5% Triton X-100 in PBS for 3 min at room temperature. Following another wash with cold PBS, the cover slips were mounted with DAPI-containing mount media (Vector Laboratories, Burlingame, Calif.) and analyzed by fluorescence microscopy.

BH3-M6, the terphenyl BH3 α-helical mimetic, and the unsubstituted terphenyl compound TPC were prepared as previously described in Kutzki, incorporated herein in its entirety by reference. (Kutzki, O., Park, H. S., Ernst, J. T., Orner, B. P., Yin, H., and Hamilton, A. D. (2002) J Am Chem Soc 124, 11838-11839)

To determine the of ability BH3-M6 to interfere with the binding of Bak to Bcl-X_{L} or Bim to Mcl-1 by fluorescence polarization the inventors used FITC-labeled peptides derived from the Bak BH3 domain (GQVGRQLAIIGDDINR) (SEQ ID NO. 3) or the Bim BH3 domain (Ahx-DMRPEIWIAQELRRIG DEFNAYYAR) (SEQ ID NO 4). (Yin, H., Lee, G. I., Sedey, K. A., Kutzki, O., Park, H. S., Orner, B. P., Ernst, J. T., Wang, H. G., Sebti, S. M., and Hamilton, A. D. (2005) J Am Chem Soc 127, 10191-10196; Oltersdorf, T., Elmore, S. W., Shoemaker, A. R., Armstrong, R. C., Augeri, D. J., Belli, B. A., Bruncko, M., Deckwerth, T. L., Dinges, J., Hajduk, P. J., Joseph, M. K., Kitada, S., Korsmeyer, S. J., Kunzer, A. R., Letai, A., Li, C., Mitten, M. J., Nettesheim, D. G., Ng, S., Nimmer, P. M., O'Connor, J. M., Oleksijew, A., Petros, A. M., Reed, J. C., Shen, W., Tahir, S. K., Thompson, C. B., Tomaselli, K. J., Wang, B., Wendt, M. D., Zhang, H., Fesik, S. W., and Rosenberg, S. H. (2005) Nature 435, 677-681; Konopleva, M., Contractor, R., Tsao, T., Samudio, I., Ruvolo, P. P., Kitada, S., Deng, X., Zhai, D., Shi, Y. X., Sneed, T., Verhaegen, M., Soengas, M., Ruvolo, V. R., McQueen, T., Schober, W. D., Watt, J. C., Jiffar, T., Ling, X., Marini, F. C., Harris, D., Dietrich, M., Estrov, Z., McCubrey, J., May, W. S., Reed, J. C., and Andreeff, M. (2006) Cancer Cell 10, 375-388)

Release of cytochrome c from isolated mitochondria was measured as described by Takahashi et al. (Takahashi, Y., Karbowski, M., Yamaguchi, H., Kazi, A., Wu, J., Sebti, S. M., Youle, R. J., and Wang, H. G. (2005) Mol Cell Biol 25, 9369-9382) Cell-free caspase-3/-7 activity and the extent of PARP cleavage was determined as described previously. (Kazi, A., Smith, D. M., Zhong, Q., and Dou, Q. P. (2002) Mol Pharmacol 62, 765-771) MTT (3-(4,5-dimethylthiazol-2-yl)-2,5-diphenyltetrazolium bromide) and TUNEL assays were performed according to Kazi et al., both of which are incorporated herein by reference. (Kazi, A., Lawrence, H., Guida, W. C., McLaughlin, M. L., Springett, G. M., Berndt, N., Yip, R. M. L., and Sebti, S. M. (2009) Cell Cycle 8, 1940-1951)

Bax and Bak expression levels were silenced by siRNA using a transfection protocol previously described by the inventors and incorporated herein by reference. (Kazi, A., Carie, A., Blaskovich, M. A., Bucher, C., That, V., Moulder, S., Peng, H., Carrico, D., Pusateri, E., Pledger, W. J., Berndt, N., Hamilton, A. D., and Sebti, S. M. (2009) Mol Cell Biol 29, 2254-2263)

### Results

Docking of BH3-M6 to Bcl-X_{L}, Bcl-2 and Mcl-1

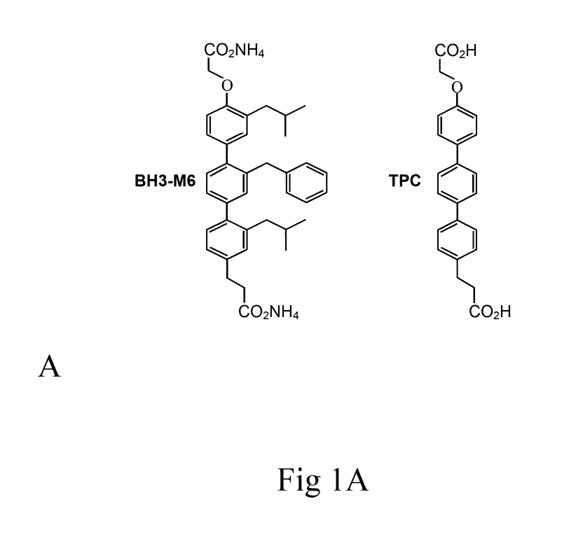

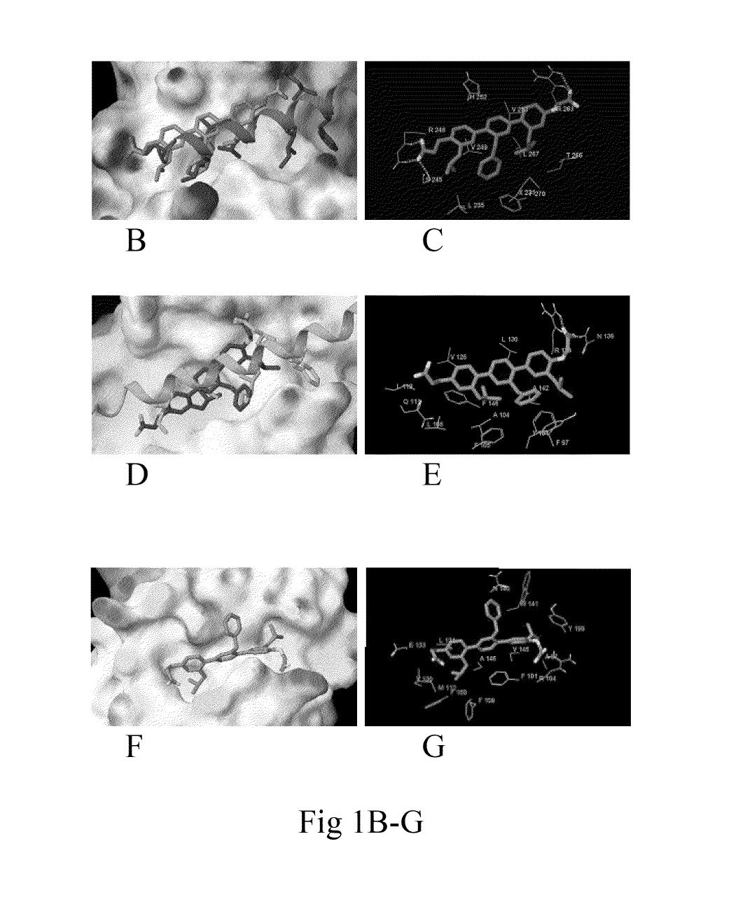

The BH3 α-helical mimic BH3-M6 and the corresponding unsubstituted terphenyl TPC (FIG. 1A) were docked to Mcl-1, Bcl-X_{L} and Bcl-2 (FIG. 1B-G). The computational docking studies suggest that the BH3-M6 head carboxylate group is near the D67 position of human Bim BH3 α-helix and forms hydrogen bonds with R263, the same human Mcl-1 residue that binds Bim D67. However, BH3-M6 does not interact with the hydrophobic pockets on Mcl-1 that bind 165 and F69 of the Bim BH3 α-helix. The computational docking studies suggest that one of the isobutyl groups of BH3-M6 is in the position of L62 of Bim and interacts with the hydrophobic pocket formed by Mcl-1 residues M231 (X231 in the figure, as modified Met in the crystal structure obtained from the PDB file), V253, L267, and F270. Although Bim L62 binds to the same hydrophobic pocket, the L62 side chain reaches into this pocket deeper than the BH3-M6 isobutyl side chain. The benzyl substituent of BH3-M6 mimics the side chain of 158 of Bim interacting with L235, and V249 of Mcl-1. The additional isobutyl lies in close proximity to L235 and V249. Finally, the tail terminal carboxylate group forms hydrogen bonds with S245 and 8248 that further stabilize the complex. The relative binding energy of BH3-M6 to Mcl-1 was about −11.3 kcal/mole. In contrast, docking of the control TPC, which lacks hydrophobic side chains, is significantly less favorable (by about 4.3 kcal/mole) than that of BH3-M6, consistent with experimental data (see below).

The BH3-M6 head carboxylate group is in the same position as D99 of the mouse Bim helix and forms hydrogen bonds with N136 and R139 of mouse Bcl-X_{L} (FIG. 1C). However, the docking results suggest that BH3-M6 does not interact significantly with the hydrophobic pocket on Bcl-X_{L} that binds F101 of the Bim BH3 α-helix. The first BH3-M6 isobutyl group is near Bcl-X_{L} residues F97 and Y101. The BH3-M6 benzyl group of BH3-M6 mimics the 197 of Bim and interacts with the hydrophobic pocket of Bcl-X_{L} that is formed by F97, Y101, and A104, the same residues that bind Bim 197. The other isobutyl group is in the position of L94 of Bim and interacts with the hydrophobic pocket of Bcl-X_{L} that is formed by F105, V126, and F146. The hydrophobic part of the propionic acid substituent is in the position of 190 of Bim and rests near hydrophobic residues L108 and L112. The relative binding energy of BH3-M6 to Bcl-X_{L} was about −10.4 kcal/mole. As in the previous example, docking of the control compound TPC is significantly less favorable (by about 4.1 kcal/mole) than that of BH3-M6, consistent with experimental data (see below).

The BH3-M6 head carboxylate group lies near human Bcl-2 residue R104 but too far to interact through hydrogen bonding (FIG. 1D). The first BH3-M6 isobutyl side chain is inserted into the binding pocket formed by Bcl-2 residues A97, W141, and Y199. The BH3-M6 benzyl side group is located near residues N140 and W141. The last BH3-M6 isobutyl side chain is in the hydrophobic pocket of F109, V130, L134, and F150. Finally, the BH3-M6 carboxylate tail is projected near residues M112 and E133. The relative binding energy of BH3-M6 to Bcl-2 was about −6.3 kcal/mole. As in the previous examples, TPC docking is significantly less favorable (by about 3.6 kcal/mole) than that of BH3-M6, consistent with experimental data (see below).

### BH3-M6 Inhibits the Binding of FITC-Bak-BH3 Peptide to Bcl-X_{L} and FITC-Bim-BH3 Peptide to Mcl-1 In Vitro and the Binding of Bcl-X_{L} and Mcl-1 to Bax and Bim in Cell-Free GST Pull-Down Assays

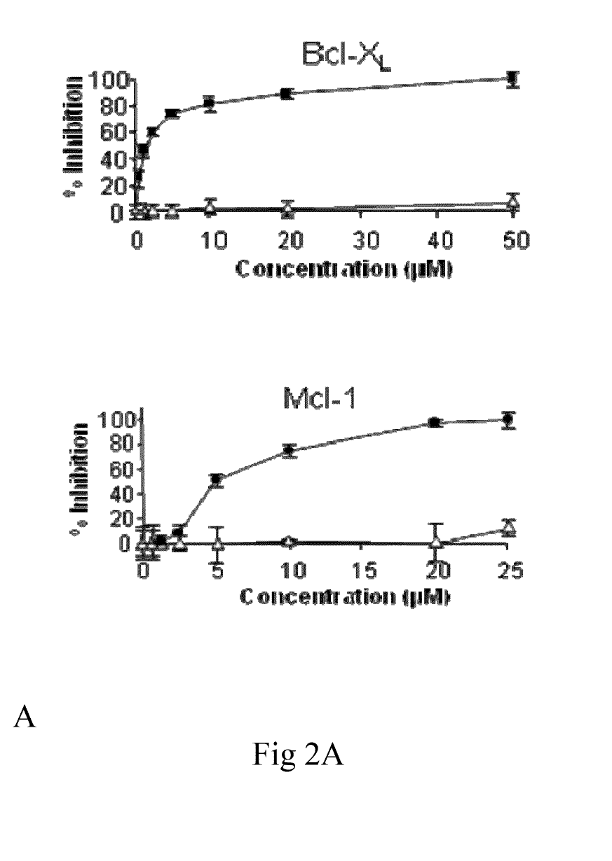

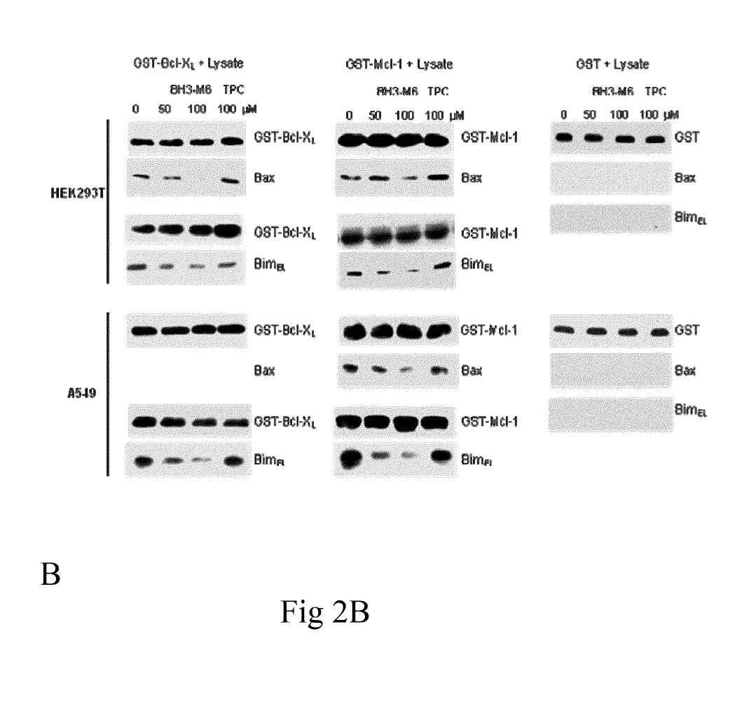

The docking studies described above show that BH3-M6 binds to anti-apoptotic proteins in a similar manner to that of the α-helix of the Bim BH3 domain. The inventors reasoned that if BH3-M6 binds anti-apoptotic proteins in this manner, then it should disrupt their binding to pro-apoptotic proteins. To evaluate this possibility, the inventors first determined whether BH3-M6 disrupts the binding of Bcl-X_{L} and Mcl-1 to Bak or Bim in vitro. Using fluorescence polarization assays as previously described (Yin, H., Lee, G. I., Sedey, K. A., Kutzki, 0., Park, H. S., Orner, B. P., Ernst, J. T., Wang, H. G., Sebti, S. M., and Hamilton, A. D. (2005) J Am Chem Soc 127, 10191-10196), the inventors showed that BH3-M6 inhibited the interaction between a FITC-labeled Bak-BH3 peptide and GST-Bcl-X_{L} or a FITC-labeled Bim-BH3 peptide and GST-Mcl-1 in a dose-dependent manner with IC₅₀ values of about 1.5 or about 4.9 μM, respectively, whereas control TPC showed lack of displacement (FIG. 2A).

The inventors next determined whether in lysates from HEK293T or A549 cells, BH3-M6 was able to disrupt the interaction between full-length Bax and Bim to full-length Bcl-X_{L} and Mcl-1, respectively, in GST pull-down assays. FIG. 2B (left panels) shows that pre-incubation of the GST-Bcl-X_{L} beads with BH3-M6 resulted in a dose-dependent inhibition of the binding of Bcl-X_{L} to Bax and Bim in both HEK293T and A549 cell lysates. In contrast, pre-incubation of the beads with the unsubstituted control TPC did not inhibit binding of Bcl-X_{L} to Bax or Bim (FIG. 2B, left panels). Similar results were observed with Mcl-1, where BH3-M6, but not TPC, disrupted the binding of Mcl-1 to Bax and Bim from HEK293T and A549 cells (FIG. 2B, middle panels). As expected the GST-only beads did not bind Bax or Bim from HEK293T or A-549 cell lysates (FIG. 2B, right panel)

BH3-M6 Disrupts Complex Formation between Pro- and Anti-Apoptotic Proteins in Intact Cells

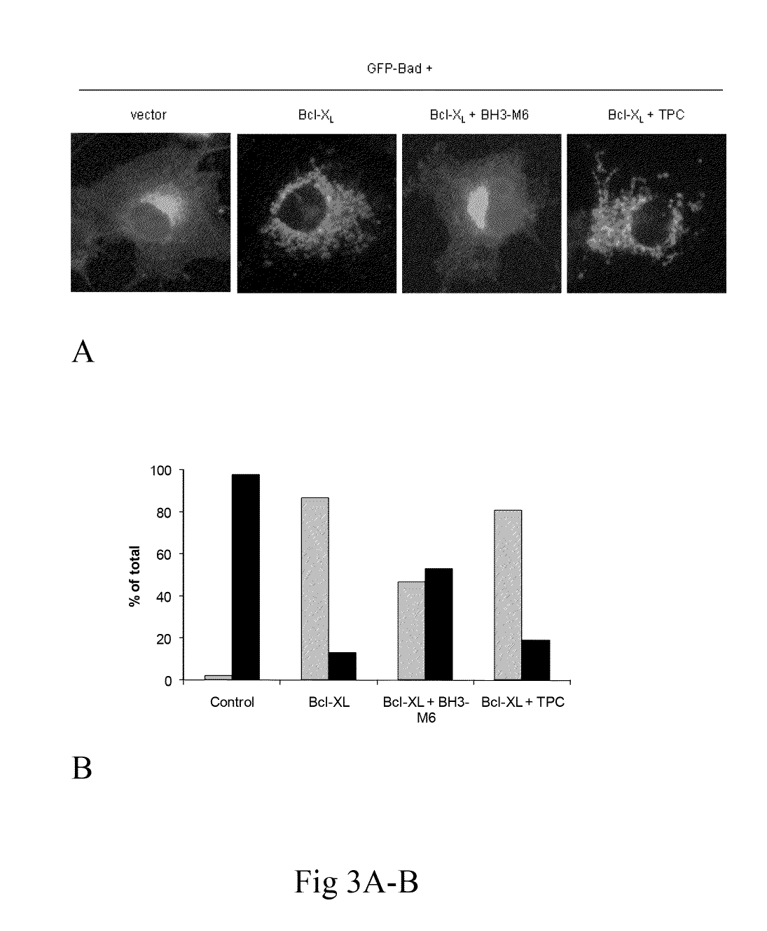

To determine whether BH3-M6 is active in intact cells, the inventors used two methods: fluorescence microscopy for cells exogenously expressing Bcl-X_{L} and GFP-Bad, as well as co-immunoprecipitation of endogenous proteins. First, as shown in FIG. 3, exogenous expression of GFP-Bad alone in COS-7 cells resulted in a diffuse pattern of fluorescence. In contrast, co-expression of GFP-Bad with HA-Bcl-X_{L} resulted, as expected, in a punctate pattern of Bad suggesting that Bcl-X_{L} bound Bad and localized it to the mitochondria. (Degterev, A., Lugovskoy, A., Cardone, M., Mulley, B., Wagner, G., Mitchison, T., and Yuan, J. (2001) Nat Cell Biol 3, 173-182) BH3-M6 treatment of COS-7 cells co-expressing GFP-Bad and Bcl-X_{L} induced a diffuse pattern suggesting that BH3-M6 was able to penetrate intact cells and inhibit Bcl-X_{L}/Bad interaction, preventing Bad from localizing into the mitochondria (FIG. 3, upper panel). Quantification of cells with diffuse vs. punctate patterns indicated that BH3-M6, but not TPC, inhibited Bcl-X_{L}-induced Bad mitochondrial localization by about 50% (FIG. 3, lower panel).

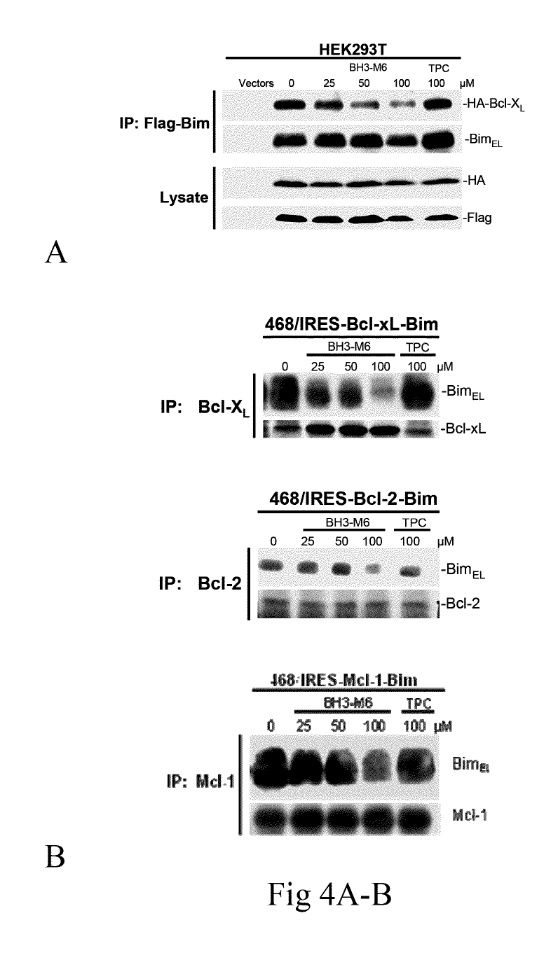

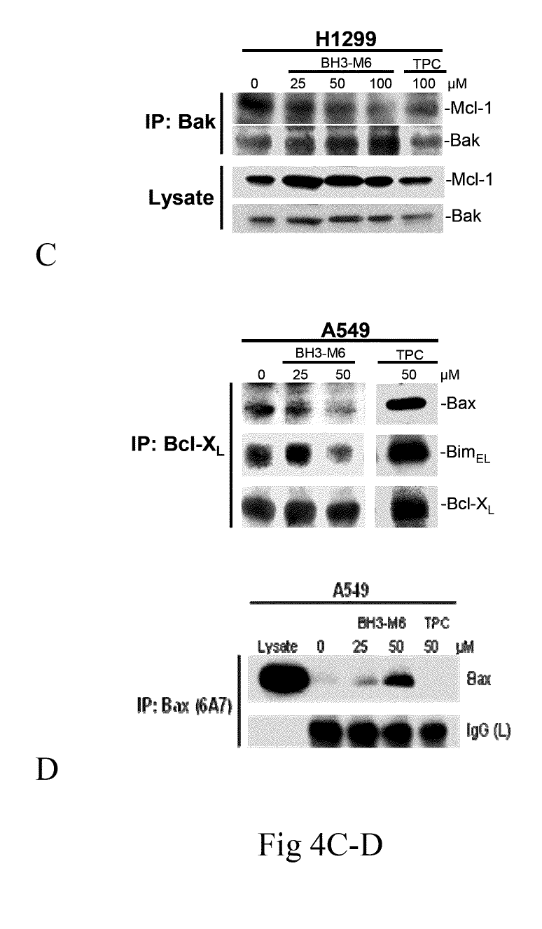

The second approach involved co-immunoprecipitation of exogenously as well as endogenously expressed proteins. First, in HEK293T cells co-transfected with HA-Bcl-X_{L} and Flag-Bim, BH3-M6, but not TPC, inhibited the association of Flag-Bim with HA-Bcl-X_{L} in a dose-dependent manner (FIG. 4A). Similarly, in human breast cancer MDA-MB-468 cells stably co-expressing either Bcl-X_{L} and Bim, Bcl-2 and Bim or Mcl-1 and Bim, BH3-M6, but not TPC, disrupted the interaction of Bim with Bcl-X_{L}, Bcl-2 and Mcl-1 in a dose-dependent manner (FIG. 4B). Taken together, the results obtained by two different approaches (FIGS. 3-4) show that BH3-M6 disrupts the interactions of exogenously expressed pro- with anti-apoptotic proteins in intact cells.

Next the inventors determined whether BH3-M6 could disrupt these interactions of the corresponding endogenous proteins. Treatment of H1299 cells with BH3-M6, but not TPC, resulted in a dose-dependent inhibition of the interaction of endogenous Bak with endogenous Mcl-1 (FIG. 4C). Likewise, in A549 cells BH3-M6 was able to disrupt the interaction of Bcl-X_{L}/Bax and Bcl-X_{L}/Bim (FIG. 4D, upper panel). Finally, a mechanism by which freed Bax is believed to induce apoptosis is through a conformational change, which can be detected by a specific antibody. Treatment of A549 cells with BH3-M6, but not TPC, increased the levels of Bax in its pro-apoptotic conformation (FIG. 4D, lower panel).

### BH3-M6 Induces Cytochrome c Release, Caspase-3/-7 Activation, Cell Death and Apoptosis

FIGS. 1-4 provide evidence that BH3-M6 disrupts the binding of pro-apoptotic to anti-apoptotic proteins in cell-free systems and in intact cells. The inventors next determined if this disruption results in triggering apoptosis. Since cytochrome c release from mitochondria to the cytosol and subsequent activation of caspases represent key steps during intrinsic apoptosis, the inventors first determined whether BH3-M6 affected this process in A549 cells.

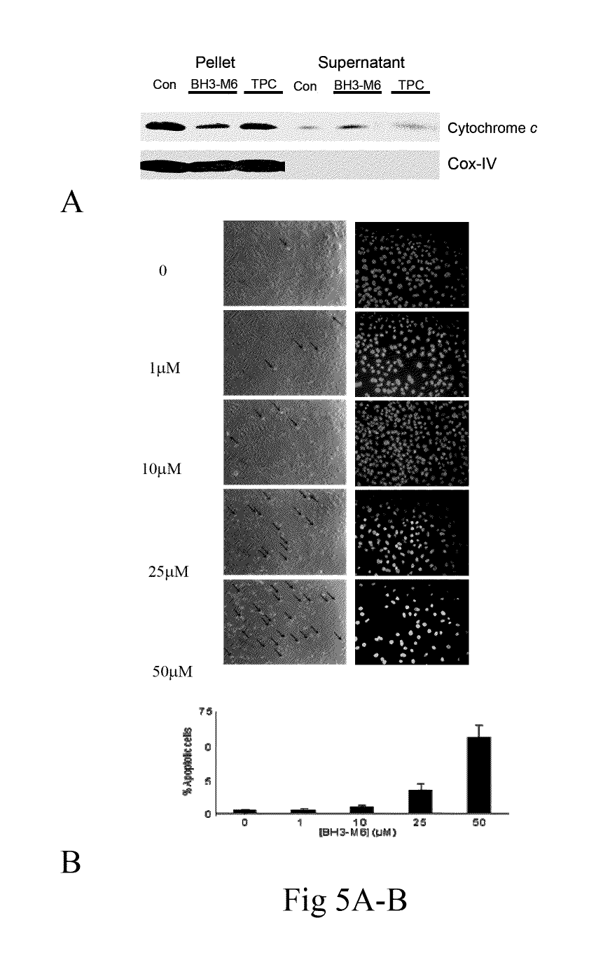

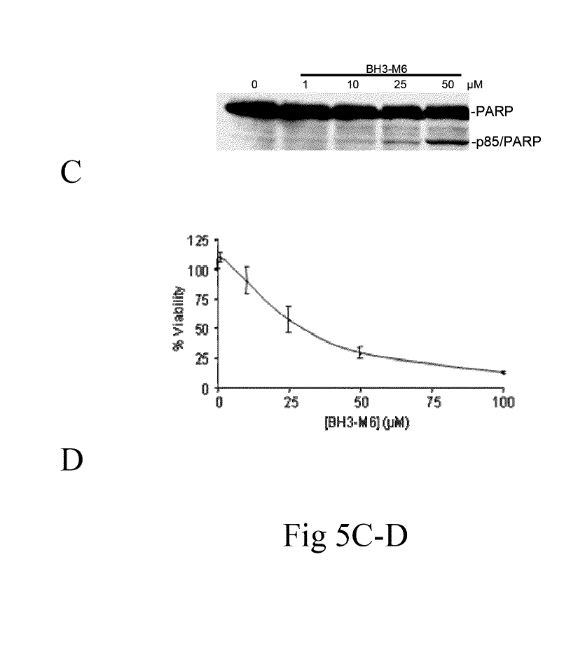

FIG. 5A shows that BH3-M6, but not TPC, triggered the release of cytochrome c from isolated mitochondria. In addition, BH3-M6 induced apoptosis as measured by DAPI staining (FIG. 5B) and the appearance of an 85 kDa PARP fragment in a concentration-dependent manner (FIG. 5C). Furthermore, treatment of A549 cells with BH3-M6 induced activation of caspase-3 and -7 by about 2.7±0.1-fold (n=3) compared to controls (data not shown) and it inhibited cell viability as measured by MTT assay in a dose-dependent manner (FIG. 5D). Taken together, these data show that BH3-M6-mediated disruption of complexes between anti- and pro-apoptotic proteins releases pro-apoptotic factors to induce intrinsic apoptosis.

Next, the inventors determined whether BH3-M6 requires caspase activity to induce apoptosis. In DoHH2 cells (an EBV-negative human B-cell non-Hodgkin's lymphoma cell line), which express high levels of Bcl-2, the BH3-M6-induced caspase-3/-7 activity (˜5-fold), PARP cleavage and apoptosis (˜7.5-fold) were abrogated by a pan-caspase inhibitor (FIG. 5E). These results show that BH3-M6-mediated induction of apoptosis requires caspase activation.

### BH3-M6 Induces Apoptosis in a Bcl-X_{L}/Bim-, Bcl-2/Bim- and Mcl-1/Bim-Dependent Manner

To investigate whether BH3-M6-induced apoptosis depends on disrupting the interaction between pro- and anti-apoptotic proteins, the inventors used three MDA-MB-468 cell lines that depend for survival on the interaction between Bcl-X_{L} and Bim, Bcl-2 and Bim or Mcl-1 and Bim (in these cells, Bim induces apoptosis when it is freed from Bcl-X_{L}, Bcl-2 or Mcl-1, respectively). (Kim, H., Rafiuddin-Shah, M., Tu, H. C., Jeffers, J. R., Zambetti, G. P., Hsieh, J. J., and Cheng, E. H. (2006) Nat Cell Biol 8, 1348-1358) In MDA-MB-468 control cells, BH3-M6 induced little apoptosis as measured by caspase-3/-7 activation and TUNEL assays (FIG. 6A). However, in MDA-MB-468 cells expressing Bcl-X_{L}/Bim, Bcl-2/Bim, Mcl-1/Bim proteins, BH3-M6 induced apoptosis by about 8- to 10-fold by caspase-3/-7 activation assay (FIG. 6A, left panel), and about 7 to 12-fold by TUNEL assay (FIG. 6A, middle panel). Furthermore, BH3-M6, but not TPC, induced PARP cleavage in these three cell lines (FIG. 6B). Coupled with the fact that BH3-M6 disrupted the interaction of Bcl-X_{L}/Bim, Bcl-2/Bim, Mcl-1/Bim in these three cell lines (see FIG. 4B), the results show that BH3-M6 disrupts the binding of anti-apoptotic proteins to Bim, which once free can induce apoptosis.

### BH3-M6-Induced Apoptosis Requires Bax but not Bak

To determine whether BH3-M6 requires Bax or Bak to induce apoptosis, the inventors treated LNCaP cells (an androgen-sensitive human prostate adenocarcinoma cell line) containing high levels of Bax and DU-145 cells (a human prostate cancer cell line) with undetectable Bax levels with BH3-M6; both cell lines express similar amounts of Bak (FIG. 6C). BH3-M6, but not TPC, induced PARP cleavage in LNCaP cells, but not in DU-145 cells (FIG. 6D). In addition, siRNA-mediated silencing of Bax or Bak expression in LNCaP cells showed that the absence of Bax, but not Bak, prevented BH3-M6-induced PARP cleavage (FIG. 6E). These data show that BH3-M6-induced apoptosis requires Bax, but not Bak, in LNCaP cells.

### BH3-M6 Sensitizes A549 Cells to the Proteasome Inhibitor CEP-1612

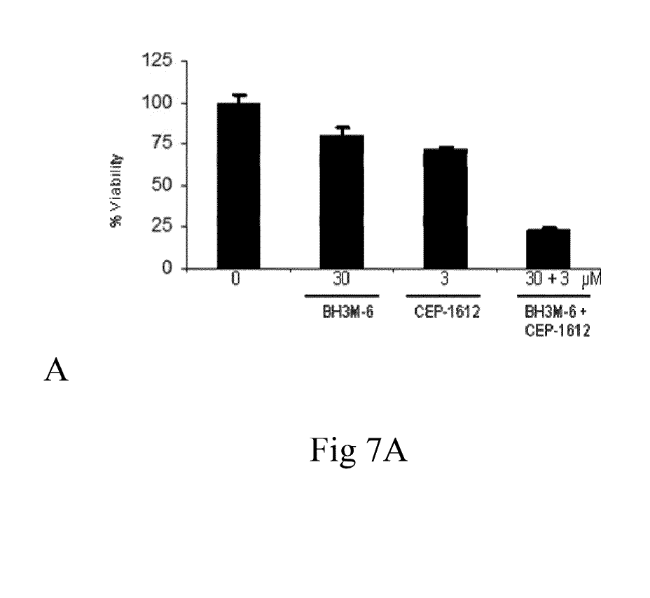

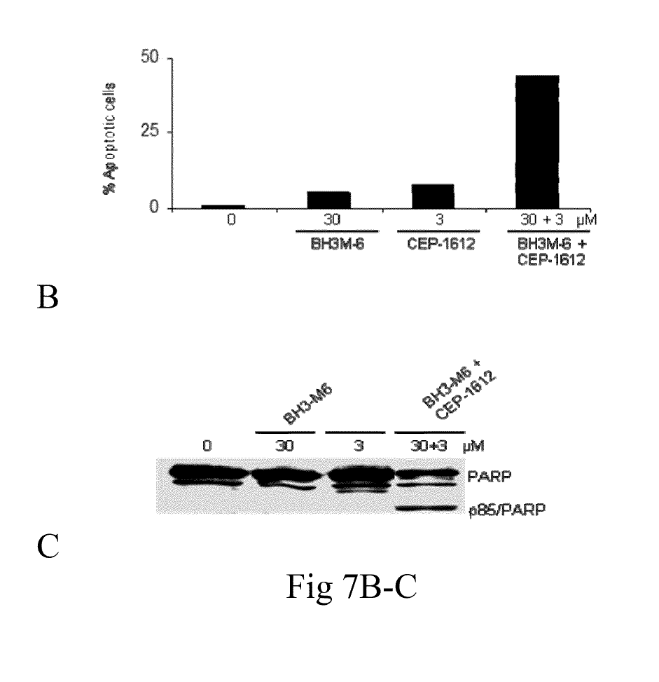

To determine whether BH3-M6 sensitizes cells to apoptosis induced by other stimuli, the inventors treated A549 cells with BH3-M6 and CEP-1612 (Sun, J., Nam, S., Lee, C. S., Li, B., Coppola, D., Hamilton, A. D., Dou, Q. P., and Sebti, S. M. (2001) Cancer Res 61, 1280-1284), either alone or in combination. BH3-M6 and CEP-1612 alone had little effect on cell viability (about 20% and about 28%, respectively) (FIG. 7A) and apoptosis (about 5% and about 8%, respectively) (FIG. 7B). However, the combination treatment showed dramatic inhibition of cell viability (about 77%) and induction of apoptosis (about 44%) (FIG. 7A, B). In addition, immunoblot analysis indicated that combination treatment, but not either drug alone induced PARP cleavage (FIG. 76). The results from FIG. 7C were obtained after about 24 hours of drug treatment, whereas those from FIG. 5C were obtained after about 48 hours of drug treatment.

### BH3-M6 Loaded Multiblock Copolymer Micelles

The pan-Bcl-2 inhibitor BH3-M6 described above may be encapsulated in a micelle to provide a more bioavailable therapeutic agent. Specifically, the BH3-M6 compound may be encapsulated within a micelle comprising a multiblock copolymer.

The multiblock copolymer comprises a hydrophilic poly(ethylene glycol) block, a carboxylic acid-containing poly(amino acid) block, and a hydrophobic D,L-mixed poly(amino acid) block characterized in that the resulting micelle has an inner core, a carboxylic acid-containing outer core, and a hydrophilic shell. It will be appreciated that the hydrophilic poly(ethylene glycol) block corresponds to the hydrophilic shell, stabilizing carboxylic acid-containing poly(amino acid) block corresponds to the carboxylic acid-containing outer core, and the hydrophobic D,L-mixed poly(amino acid) block corresponds to the inner core.

Amphiphilic multiblock copolymers, as described herein, can self-assemble in aqueous solution to form nano- and micron-sized structures. In water, these amphiphilic multiblock copolymers assemble by multi-molecular micellization when present in solution above the critical micelle concentration (CMC). Without wishing to be bound by any particular theory, it is believed that the hydrophobic poly(amino acid) portion or “block” of the copolymer collapses to form the micellar core, while the hydrophilic PEG block forms a peripheral corona and imparts water solubility. In certain embodiments, the multiblock copolymers in accordance with the present invention possess distinct hydrophobic and hydrophilic segments that form micelles. In addition, these multiblock polymers optionally comprise a poly(amino acid) block which contains functionality suitable for crosslinking. It will be appreciated that this functionality is found on the corresponding amino acid side-chain.

In certain embodiments, the present invention provides a micelle having BH3-M6 encapsulated therein, wherein said micelle comprises a multiblock copolymer which comprises: a hydrophilic poly(ethylene glycol) block; a stabilizing carboxylic acid-containing poly(amino acid) block; and a hydrophobic D,L-mixed poly(amino acid) block.

In some embodiments, the stabilizing carboxylic acid-containing poly(amino acid) block is a poly(glutamic acid) block or a poly(aspartic acid) block. In other embodiments, the stabilizing carboxylic acid-containing poly(amino acid) block is a random poly(glutamic acid-co-apartic acid) block.

The “hydrophobic D,L-mixed poly(amino acid)” block, as described herein, consists of a mixture of D and L enantiomers to facilitate the encapsulation of hydrophobic moieties. It is well established that homopolymers and copolymers of amino acids, consisting of a single stereoisomer, may exhibit secondary structures such as the α-helix or β-sheet. (H. R. Kricheldorf, α-Aminoacid-N-Caroboxy-Anhydrides and Related Heterocycles, Springer-Verlag, 1987). For example, poly(L-benzyl glutatmate) typically exhibits an α-helical conformation; however this secondary structure can be disrupted by a change of solvent or temperature. (P. Urnes and P. Doty, Advances in Protein Chemistry XVI, Academic Press, New York 1961). The secondary structure can also be disrupted by the incorporation of structurally dissimilar amino acids such as b-sheet forming amino acids (e.g. proline) or through the incorporation of amino acids with dissimilar stereochemistry (e.g. mixture of D and L stereoisomers), which results in poly(amino acids) with a random coil conformation. (Sakai, R.; Ikeda; S.; Isemura, T. Bull Chem. Soc. Japan 1969, 42, 1332-1336, Paolillo, L.; Temussi, P. A.; Bradbury, E. M.; Crane-Robinson, C. Biopolymers 1972, 11, 2043-2052, and Cho, I.; Kim, J. B.; Jung, H. J. Polymer 2003, 44, 5497-5500).

While the methods to influence secondary structure of poly(amino acids) have been known for some time, it has been surprisingly discovered that block copolymers of the present invention, possessing a random coil conformation, are particularly useful for encapsulation of hydrophobic molecules, especially BH3-M6, when compared to similar block copolymers possessing a helical segment. Without wishing to be bound to any particular theory, it is believed that the provided block copolymers having a coil-coil conformation allow for efficient packing and loading of hydrophobic moieties within the micelle core, while the steric demands of a rod-coil conformation for a helix-containing block copolymer results in less effective encapsulation. Indeed, it has been found that encapsulation of BH3-M6 within a provided copolymer micelle allows for drastically increased solubility of BH3-M6 in water. As used herein, the term “free BH3-M6” refers to BH3-M6 that is not encapsulated by a provided micelle in accordance with the present invention.

In certain embodiments, the PEG block possesses a molecular weight of about 10,000 Da (225 repeat units). In other embodiments, the PEG block possesses a molecular weight of about 12,000 Da (270 repeat units). In yet other embodiment, the PEG block possesses a molecular weight of about 8,000 Da (180 repeat units). In certain embodiments, the PEG block possesses a molecular weight of about 20,000 Da (450 repeat units). Without wishing to be bound by theory, it is believed that this particular PEG chain length imparts adequate water-solubility to the micelles and provides relatively long in vivo circulation times.

### Formula I

In certain embodiments, the present invention provides a micelle, having BH3-M6 encapsulated therein, comprising a multiblock copolymer of formula I:

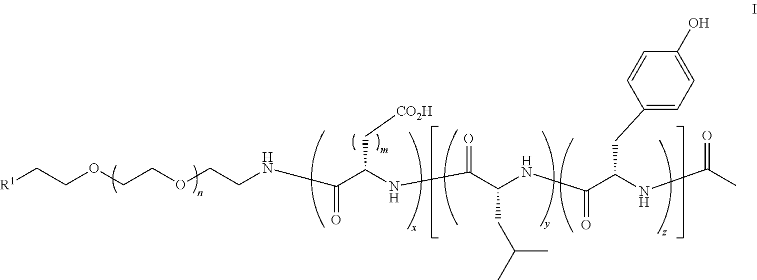

wherein:
R¹ is —OCH₃, —N₃, or

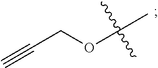 [SMILES: *OCC#C]

n is 110 to 450;
m is 1 or 2;
x is 3 to 50;
y is 5 to 50; and
z is 5 to 50.

In certain embodiments, the present invention provides a micelle, having BH3-M6 encapsulated therein, comprising a multiblock copolymer of formula I:

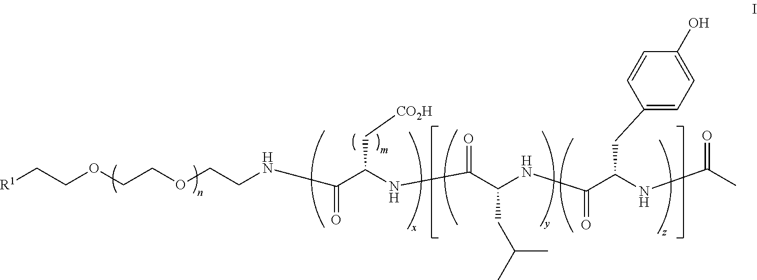

wherein:
R¹ is —N₃;
n is about 270;
m is 1;
x is 10-20;
y is about 20; and
z is about 20.

In certain embodiments, the present invention provides a micelle, having BH3-M6 encapsulated therein, comprising a multiblock copolymer of formula I:

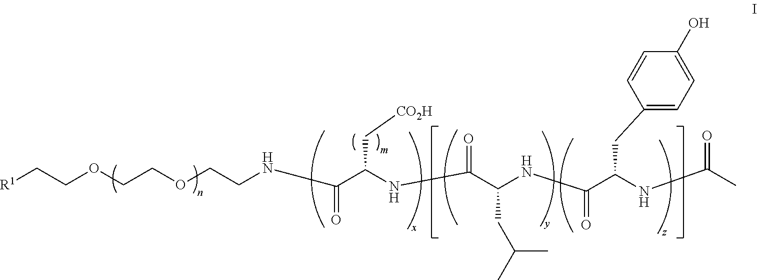

wherein:
R¹ is —OCH₃;
n is about 270;
m is 1;
x is 10-20;
y is about 20; and
z is about 20.

As defined generally above, the n group of formula I is 110-450. In certain embodiments, the present invention provides compounds of formula I, as described above, wherein n is about 225. In other embodiments, n is about 270. In other embodiments, n is about 350. In other embodiments, n is about 110. In other embodiments, n is about 450. In other embodiments, n is selected from 110±10, 180±10, 225±10, 275±10, 315±10, or 450±10.

As defined generally above, the m group of formula I is 1 or 2. In some embodiments, m is 1 thereby forming a poly(aspartic acid) block. In some embodiments, m is 2 thereby forming a poly(glutamic acid) block.

In certain embodiments, the x group of formula I is about 3 to about 50. In certain embodiments, the x group of formula I is about 10. In other embodiments, x is about 20. According to yet another embodiment, x is about 15. In other embodiments, x is about 5. In other embodiments, x is selected from 5±3, 10±3, 10±5, 15±5, or 20±5.

In certain embodiments, the y group of formula I is about 5 to about 50. In certain embodiments, the y group of formula I is about 10. In other embodiments, y is about 20. According to yet another embodiment, y is about 15. In other embodiments, y is about 30. In other embodiments, y is selected from 10±3, 15±3, 17±3, 20±5, 30±5, or 40±5.

In certain embodiments, the z group of formula I is about 5 to about 50. In certain embodiments, the z group of formula I is about 10. In other embodiments, z is about 20. According to yet another embodiment, z is about 15. In other embodiments, z is about 30. In other embodiments, z is selected from 10±3, 15±3, 17±3, 20±5, 30±5, or 40±5.

In some embodiments, the R¹ group of a compound of formula I is —N₃ suitable for Click chemistry, and therefore useful for conjugating said compound to biological systems or macromolecules such as proteins, viruses, and cells, to name but a few. The Click reaction is known to proceed quickly and selectively under physiological conditions. In contrast, most conjugation reactions are carried out using the primary amine functionality on proteins (e.g. lysine or protein end-group). Because most proteins contain a multitude of lysines and arginines, such conjugation occurs uncontrollably at multiple sites on the protein. This is particularly problematic when lysines or arginines are located around the active site of an enzyme or other biomolecule. Thus, another embodiment of the present invention provides a method of conjugating the azide end group of a compound of formula I to a macromolecule via Click chemistry. Yet another embodiment of the present invention provides a macromolecule conjugated to a compound of formula I via the R¹ azide group.

### Formula II

In certain embodiments, the present invention provides a micelle, having BH3-M6 encapsulated therein, comprising a multiblock copolymer of formula II:

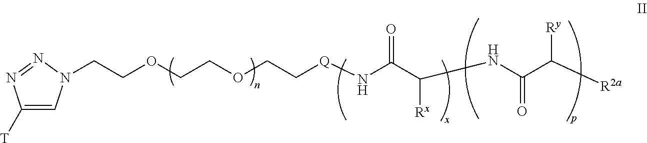

wherein:
n is 10-2500;
x is 0 to 1000;
p is 2 to 1000;
R^{x} is a natural or unnatural amino acid side-chain group that is capable of crosslinking;
R³′ forms a hydrophobic D,L-mixed poly(amino acid) block;
Q is a valence bond or a bivalent, saturated or unsaturated, straight or branched C₁₋₁₂ hydrocarbon chain, wherein 0-6 methylene units of Q are independently replaced by -Cy-, —O—, —NH—, —S—, —OC(O)—, —C(O)O—, —C(O)—, —SO—, —SO₂—, —NHSO₂—, —SO₂NH—, —NHC(O)—, —C(O)NH—, —OC(O)NH—, or —NHC(O)O—, wherein:
   -Cy- is an optionally substituted 5-8 membered bivalent, saturated, partially unsaturated, or aryl ring having 0-4 heteroatoms independently selected from nitrogen, oxygen, or sulfur, or an optionally substituted 8-10 membered bivalent saturated, partially unsaturated, or aryl bicyclic ring having 0-5 heteroatoms independently selected from nitrogen, oxygen, or sulfur;
R^{2a} is a mono-protected amine, a di-protected amine, —N(R⁴)₂, —NR⁴C(O)R⁴, —NR⁴C(O)N(R⁴)₂, —NR⁴C(O)OR⁴, or —NR⁴SO₂R⁴;
each R⁴ is independently hydrogen or an optionally substituted group selected from aliphatic, a 5-8 membered saturated, partially unsaturated, or aryl ring having 0-4 heteroatoms independently selected from nitrogen, oxygen, or sulfur, an 8-10 membered saturated, partially unsaturated, or aryl bicyclic ring having 0-5 heteroatoms independently selected from nitrogen, oxygen, or sulfur, or a detectable moiety, or:
   two R⁴ on the same nitrogen atom are taken together with said nitrogen atom to form an optionally substituted 4-7 membered saturated, partially unsaturated, or aryl ring having 1-4 heteroatoms independently selected from nitrogen, oxygen, or sulfur, and
T is a targeting group moiety.

In certain embodiments, the p group of formula II is about 5 to about 500. In certain embodiments, the p group of formula II is about 10 to about 250. In other embodiments, p is about 10 to about 50. According to yet another embodiment, p is about 15 to about 40. In other embodiments, p is about 20 to about 40. According to yet another embodiment, p is about 50 to about 75. According to other embodiments, x and p are independently about 10 to about 100.

In some embodiments, x is 0. In certain embodiments, x is 5-50. In other embodiments, x is 5-25. In certain embodiments, p is 5-50. In other embodiments, p is 5-10. In other embodiments, p is 10-20. In certain embodiments, x and p add up to about 30 to about 60. In still other embodiments, x is 1-20 repeat units and p is 10-50 repeat units. In certain embodiments, the x group of formula II is about 3 to about 50. In certain embodiments, the x group of formula II is about 10. In other embodiments, x is about 20. According to yet another embodiment, x is about 15. In other embodiments, x is about 5. In other embodiments, x is selected from 5±3, 10±3, 10±5, 15±5, or 20±5.

As defined generally above, the Q group of formula II is a valence bond or a bivalent, saturated or unsaturated, straight or branched C₁₋₁₂ hydrocarbon chain, wherein 0-6 methylene units of Q are independently replaced by -Cy-, —O—, —NH—, —S—, —OC(O)—, —C(O)O—, —C(O)—, —SO—, —SO₂—, —NHSO₂—, —SO₂NH—, —NHC(O)—, —C(O)NH—, —OC(O)NH—, or —NHC(O)O—, wherein -Cy- is an optionally substituted 5-8 membered bivalent, saturated, partially unsaturated, or aryl ring having 0-4 heteroatoms independently selected from nitrogen, oxygen, or sulfur, or an optionally substituted 8-10 membered bivalent saturated, partially unsaturated, or aryl bicyclic ring having 0-5 heteroatoms independently selected from nitrogen, oxygen, or sulfur. In certain embodiments, Q is a valence bond. In other embodiments, Q is a bivalent, saturated Cl_{—}12 alkylene chain, wherein 0-6 methylene units of Q are independently replaced by -Cy-, —O—, —NH—, —S—, —OC(O)—, —C(O)O—, or —C(O)—, wherein -Cy- is an optionally substituted 5-8 membered bivalent, saturated, partially unsaturated, or aryl ring having 0-4 heteroatoms independently selected from nitrogen, oxygen, or sulfur, or an optionally substituted 8-10 membered bivalent saturated, partially unsaturated, or aryl bicyclic ring having 0-5 heteroatoms independently selected from nitrogen, oxygen, or sulfur.

In certain embodiments, the Q group of formula II is -Cy- (i.e. a C₁₋alkylene chain wherein the methylene unit is replaced by -Cy-), wherein -Cy- is an optionally substituted 5-8 membered bivalent, saturated, partially unsaturated, or aryl ring having 0-4 heteroatoms independently selected from nitrogen, oxygen, or sulfur. According to one aspect of the present invention, -Cy- is an optionally substituted bivalent aryl group. According to another aspect of the present invention, -Cy- is an optionally substituted bivalent phenyl group. In other embodiments, -Cy- is an optionally substituted 5-8 membered bivalent, saturated carbocyclic ring. In still other embodiments, -Cy- is an optionally substituted 5-8 membered bivalent, saturated heterocyclic ring having 1-2 heteroatoms independently selected from nitrogen, oxygen, or sulfur. Exemplary -Cy-groups include bivalent rings selected from phenyl, pyridyl, pyrimidinyl, cyclohexyl, cyclopentyl, or cyclopropyl.

In certain embodiments, the R^{x} group of formula II is a crosslinkable amino acid side-chain group. Such crosslinkable amino acid side-chain groups include tyrosine, serine, cysteine, threonine, aspartic acid (also known as aspartate, when charged), glutamic acid (also known as glutamate, when charged), asparagine, histidine, lysine, arginine, glutamine, or a benzimidazole-functionalized amino acid.

As defined above, the R^{x} group of formula II is a natural or unnatural amino acid side-chain group capable of forming crosslinks. It will be appreciated that a variety of amino acid side-chain functional groups are capable of such crosslinking, including, but not limited to, carboxylate, hydroxyl, thiol, and amino groups. Examples of R^{x} moieties having functional groups capable of forming crosslinks include a glutamic acid side-chain, —CH₂C(O)OH, an aspartic acid side-chain, —CH₂CH₂C(O)OH, a cysteine side-chain, —CH₂SH, a serine side-chain, —CH₂OH, an aldehyde containing side-chain, —CH₂C(O)H, a lysine side-chain, —(CH₂)₄NH₂, an arginine side-chain, —(CH₂)₃NHC(═NH)NH₂, a histidine side-chain, —CH₂-imidazol-4-yl. In some embodiments, R^{x} is a glutamic acid side chain. In other embodiments, R^{x} is an aspartic acid side chain. In still other embodiments, R^{x} is a histidine side-chain.

As defined above, the R^{y} group of formula II forms a hydrophobic D,L-mixed amino acid block. Such hydrophobic amino acid side-chain groups include a suitably protected tyrosine side-chain, a suitably protected serine side-chain, a suitably protected threonine side-chain, phenylalanine, alanine, valine, leucine, tryptophan, proline, benzyl and alkyl glutamates, or benzyl and alkyl aspartates or mixtures thereof. One of ordinary skill in the art would recognize that protection of a polar or hydrophilic amino acid side-chain can render that amino acid nonpolar. For example, a suitably protected tyrosine hydroxyl group can render that tyrosine nonpolar and hydrophobic by virtue of protecting the hydroxyl group. Suitable protecting groups for the hydroxyl, amino, and thiol, and carboylate functional groups of R^{x} and R^{y} are as described herein.

In other embodiments, the R^{y} group of formula II consists of a mixture of D-hydrophobic and L-hydrophilic amino acid side-chain groups such that the overall poly(amino acid) block comprising R^{y} is hydrophobic and is a mixture of D- and L-configured amino acids. Such mixtures of amino acid side-chain groups include L-tyrosine and D-leucine, L-tyrosine and D-phenylalanine, L-serine and D-phenylalanine, L-aspartic acid and D-phenylalanine, L-glutamic acid and D-phenylalanine, L-tyrosine and D-benzyl glutamate, L-tyrosine and D-benzyl aspartate, L-serine and D-benzyl glutamate, L-serine and D-benzyl aspartate, L-aspartic acid and D-benzyl glutamate, L-aspartic acid and D-benzyl aspartate, L-glutamic acid and D-benzyl glutamate, L-glutamic acid and D-benzyl aspartate, L-aspartic acid and D-leucine, and L-glutamic acid and D-leucine. Ratios (D-hydrophobic to L-hydrophilic) of such amino acid combinations can range between 5-95 mol %.

In certain embodiments, the R^{y} group of formula II consists of a mixture of D-hydrophobic and L-hydrophobic amino acids. Such mixtures include D-benzyl glutamate and L-benzyl glutamate, D-benzyl aspartate and L-benzyl aspartate, D-benzyl aspartate and L-benzyl glutamate, or D-benzyl glutamate and L-benzyl aspartate.

As defined generally above, the R^{2a} group of formula II is a mono-protected amine, a di-protected amine, —NHR⁴, —N(R⁴)₂, —NHC(O)R⁴, —NR⁴C(O)R⁴, —NHC(O)NHR⁴, —NHC(O)N(R⁴)₂, —NR⁴C(O)NHR⁴, —NR⁴C(O)N(R⁴)₂, —NHC(O)OR⁴, —NR⁴C(O)OR⁴, —NHSO₂R⁴, or —NR⁴SO₂R⁴, wherein each R⁴ is independently an optionally substituted group selected from aliphatic, a 5-8 membered saturated, partially unsaturated, or aryl ring having 0-4 heteroatoms independently selected from nitrogen, oxygen, or sulfur, an 8-10-membered saturated, partially unsaturated, or aryl bicyclic ring having 0-5 heteroatoms independently selected from nitrogen, oxygen, or sulfur, or a detectable moiety, or two R⁴ on the same nitrogen atom are taken together with said nitrogen atom to form an optionally substituted 4-7 membered saturated, partially unsaturated, or aryl ring having 1-4 heteroatoms independently selected from nitrogen, oxygen, or sulfur.

In certain embodiments, the R^{2a} group of formula II is —NHR⁴ or —N(R⁴)₂ wherein each R⁴ is an optionally substituted aliphatic group. One exemplary R⁴ group is 5-norbornen-2-yl-methyl. According to yet another aspect of the present invention, the R^{2a} group of formula I is —NHR⁴ wherein R⁴ is a C₁₋₆ aliphatic group substituted with N3. Examples include —CH₂N3. In some embodiments, R⁴ is an optionally substituted C₁₋₆ alkyl group. Examples include methyl, ethyl, propyl, butyl, pentyl, hexyl, 2-(tetrahydropyran-2-yloxy)ethyl, pyridin-2-yldisulfanylmethyl, methyldisulfanylmethyl, (4-acetylenylphenyl)methyl, 3-(methoxycarbonyl)-prop-2-ynyl, methoxycarbonylmethyl, 2-(N-methyl-N-(4-acetylenylphenyl)carbonylamino)-ethyl, 2-phthalimidoethyl, 4-bromobenzyl, 4-chlorobenzyl, 4-fluorobenzyl, 4-iodobenzyl, 4-propargyloxybenzyl, 2-nitrobenzyl, 4-(bis-4-acetylenylbenzyl)aminomethyl-benzyl, 4-propargyloxy-benzyl, 4-dipropargylamino-benzyl, 4-(2-propargyloxy-ethyldisulfanyl)benzyl, 2-propargyloxy-ethyl, 2-propargyldisulfanyl-ethyl, 4-propargyloxy-butyl, 2-(N-methyl-N-propargylamino)ethyl, and 2-(2-dipropargylaminoethoxy)-ethyl. In other embodiments, R⁴ is an optionally substituted C₂₋₆ alkenyl group. Examples include vinyl, allyl, crotyl, 2-propenyl, and but-3-enyl. When R⁴ group is a substituted aliphatic group, suitable substituents on R⁴ include N₃, CN, and halogen. In certain embodiments, R⁴ is —CH₂CN, —CH₂CH₂CN, —CH₂CH(OCH₃)₂, 4-(bisbenzyloxymethyl)phenylmethyl, and the like.

According to another aspect of the present invention, the R^{2a} group of formula II is —NHR⁴ wherein R⁴ is an optionally substituted C₂₋₆ alkynyl group. Examples include —CC≡CH, —CH₂C≡CH, —CH₂C≡CCH₃, and —CH₂CH₂C≡CH.

In certain embodiments, the R^{2a} group of formula II is —NHR⁴ wherein R⁴ is an optionally substituted 5-8-membered aryl ring. In certain embodiments, R⁴ is optionally substituted phenyl or optionally substituted pyridyl. Examples include phenyl, 4-t-butoxycarbonylaminophenyl, 4-azidomethylphenyl, 4-propargyloxyphenyl, 2-pyridyl, 3-pyridyl, and 4-pyridyl. In certain embodiments, R^{2a} is 4-t-butoxycarbonylaminophenylamino, 4-azidomethylphenamino, or 4-propargyloxyphenylamino.

In certain embodiments, the R^{2a} group of formula II is —NHR⁴ wherein R⁴ is an optionally substituted phenyl ring. Suitable substituents on the R⁴ phenyl ring include halogen; —(CH₂)₀₋₄R^{o}; —(CH₂)₀₋₄OR^{o}; —(CH₂)₀₋₄—CH(OR^{o}₂; —(CH₂)₀₋₄SR^{o}; —(CH₂)₀₋₄Ph, which may be substituted with R^{o}; —(CH₂)₀₋₄O(CH₂)₀₋₁Ph which may be substituted with R^{o}; —CH═CHPh, which may be substituted with R^{o}; —NO₂; —CN; —N3; —(CH₂)₀₋₄N(R^{o})₂; —(CH₂)₀₋₄N(R^{o}C(O)R^{o}; —N(R^{o}C(S)R^{o}; —(CH₂)₀₋₄N(R^{o}C(O)NR^{o}₂; —N(R^{o}C(S)NR^{o}₂; —(CH₂)₀₋₄N(R^{o}C(O)OR^{o}; —N(R^{o})N(R^{o}C(O)R^{o}; —N(R^{o})N(R^{o}C(O)NR^{o}₂; —N(R^{o})N(R^{o}C(O)OR^{o}; —(CH₂)₀₋₄C(O)R^{o}; —C(S)R^{o}; —(CH₂)₀₋₄C(O)OR^{o}; —(CH₂)₀₋₄C(O)SR^{o}; —(CH₂)₀₋₄C(O)OSiR^{o}₃; —(CH₂)₀₋₄OC(O)R^{o}; —(CH₂)₀₋₄SC(O)R^{o}; —(CH₂)₀₋₄C(O)NR^{o}₂; —C(S)NR^{o}₂; —(CH₂)₀₋₄OC(O)NR^{o}₂; —C(O)N(OR^{o})R^{o}; —C(O)C(O)R^{o}; —C(O)CH₂C(O)R^{o}; —C(NOR^{o})R^{o}; —(CH₂)₀₋₄SSR^{o}; —(CH₂)₀₋₄S(O)₂R^{o}; —(CH₂)₀₋₄S(O)₂OR^{o}; —(CH₂)₀₋₄OS(O)₂R^{o}; —S(O)₂NR^{o}₂; —(CH₂)₀₋₄S(O)R^{o}; —N(R^{o})S(O)₂NR^{o}₂; —N(R^{o}S(O)₂R^{o}; —N(OR^{o})R^{o}; —C(NH)NR^{o}₂; —P(O)₂R^{o}; —P(O)R^{o}₂; —OP(O)R^{o}₂; SiR^{o}₃; wherein each independent occurrence of R^{o} is as defined herein supra. In other embodiments, the R^{2a} group of formula I is —NHR⁴ wherein R⁴ is phenyl substituted with one or more optionally substituted Cl_{—}6 aliphatic groups. In still other embodiments, R⁴ is phenyl substituted with vinyl, allyl, acetylenyl, —CH₂N3, —CH₂CH₂N3, —CH₂C≡CCH₃, or —CH₂C≡CH.

In certain embodiments, the R^{2a} group of formula II is —NHR⁴ wherein R⁴ is phenyl substituted with N3, N(R^{o})₂, CO₂R^{o}, or C(O)R^{o} wherein each R^{o} is independently as defined herein supra.

In certain embodiments, the R^{2a} group of formula II is —N(R⁴)₂ wherein each R⁴ is independently an optionally substituted group selected from aliphatic, phenyl, naphthyl, a 5-6 membered aryl ring having 1-4 heteroatoms independently selected from nitrogen, oxygen, or sulfur, or a 8-10 membered bicyclic aryl ring having 1-5 heteroatoms independently selected from nitrogen, oxygen, or sulfur, or a detectable moiety.

In other embodiments, the R^{2a} group of formula II is —N(R⁴)₂ wherein the two R⁴ groups are taken together with said nitrogen atom to form an optionally substituted 4-7 membered saturated, partially unsaturated, or aryl ring having 1-4 heteroatoms independently selected from nitrogen, oxygen, or sulfur. According to another embodiment, the two R⁴ groups are taken together to form a 5-6-membered saturated or partially unsaturated ring having one nitrogen wherein said ring is substituted with one or two oxo groups. Such R^{2a} groups include, but are not limited to, phthalimide, maleimide and succinimide.

In certain embodiments, the R^{2a} group of formula II is a mono-protected or di-protected amino group. In certain embodiments R^{2a} is a mono-protected amine. In certain embodiments R^{2a} is a mono-protected amine selected from aralkylamines, carbamates, allyl amines, or amides. Exemplary mono-protected amino moieties include t-butyloxycarbonylamino, ethyloxycarbonylamino, methyloxycarbonylamino, trichloroethyloxy-carbonylamino, allyloxycarbonylamino, benzyloxocarbonylamino, allylamino, benzylamino, fluorenylmethylcarbonyl, formamido, acetamido, chloroacetamido, dichloroacetamido, trichloroacetamido, phenylacetamido, trifluoroacetamido, benzamido, and t-butyldiphenylsilylamino. In other embodiments R^{2a} is a di-protected amine. Exemplary di-protected amino moieties include di-benzylamino, di-allylamino, phthalimide, maleimido, succinimido, pyrrolo, 2,2,5,5-tetramethyl-[1,2,5]azadisilolidino, and azido. In certain embodiments, the R^{2a} moiety is phthalimido. In other embodiments, the R^{2a} moiety is mono- or di-benzylamino or mono- or di-allylamino.

As defined generally above, the T group of formula II is a targeting group moiety. Targeting groups are well known in the art and include those described in International Application Publication Number WO2008/134761, published Nov. 6, 2008, the entirety of which is hereby incorporated by reference. In some embodiments, the T targeting group is a moiety selected from folate, a Her-2 binding peptide, a urokinase-type plasminogen activator receptor (uPAR) antagonist, a CXCR4 chemokine receptor antagonist, a GRP78 peptide antagonist, an RGD peptide, an RGD cyclic peptide, a luteinizing hormone-releasing hormone (LHRH) antagonist peptide, an aminopeptidase targeting peptide, a brain homing peptide, a kidney homing peptide, a heart homing peptide, a gut homing peptide, an integrin homing peptide, an angiogencid tumor endothelium homing peptide, an ovary homing peptide, a uterus homing peptide, a sperm homing peptide, a microglia homing peptide, a synovium homing peptide, a urothelium homing peptide, a prostate homing peptide, a lung homing peptide, a skin homing peptide, a retina homing peptide, a pancreas homing peptide, a liver homing peptide, a lymph node homing peptide, an adrenal gland homing peptide, a thyroid homing peptide, a bladder homing peptide, a breast homing peptide, a neuroblastoma homing peptide, a lymphoma homing peptide, a muscle homing peptide, a wound vasculature homing peptide, an adipose tissue homing peptide, a virus binding peptide, or a fusogenic peptide. Such targeting groups are well known in the art and are described in detail in WO 2008/134761.

In some embodiments, the T targeting group is a moiety selected from a tumor homing group, a prostate specific membrane antigen homing peptide, an aminopeptidate N homing peptide, a Her-2 homing peptide, a colon cancer homing peptide, a VEGFR1 homing peptide, or a CXCR4 homing peptide.

In certain embodiments, the present invention provides a micelle, having BH3-M6 encapsulated therein, comprising a multiblock copolymer of formula II, as defined above and described herein.

In some embodiments, the present invention provides a micelle, having BH3-M6 encapsulated therein, comprising a multiblock copolymer of formula I and a multiblock copolymer of formula II, wherein each of formula I and formula II are as defined above and described herein, wherein the ratio of Formula I to Formula II is between about 1000:1 and about 1:1. In other embodiments, the ratio is about 1000:1, about 100:1, about 50:1, about 33:1, about 25:1, about 20:1, about 10:1, about 5:1, or about 4:1. In yet other embodiments, the ratio is between about 100:1 and about 25:1.

### Crosslinked BH3-M6 Loaded Micelles

Crosslinking reactions designed for drug delivery preferably meet a certain set of requirements to be deemed safe and useful for in vivo applications. For example, in other embodiments, the crosslinking reaction could utilize non-cytotoxic reagents, would be insensitive to water, would not alter the drug to be delivered, and in the case of cancer therapy, would be reversible at pH levels commonly encountered in tumor tissue (pH ˜6.8) or acidic organelles in cancer cells (pH ˜5.0-6.0).

In certain embodiments, the crosslinking chemistry utilizes zinc-mediated coupling of carboxylic acids, a highly selective and pH-sensitive reaction that is performed in water. This reaction, which is widely used in cough lozenge applications, involves the association of zinc ions with carboxylic acids at basic pH. (Bakar, N. K. A.; Taylor, D. M.; Williams, D. R. Chem. Spec. Bioavail. 1999, 11, 95-101; and Eby, G. A. J. Antimicrob. Chemo. 1997, 40, 483-493). These zinc-carboxylate bonds readily dissociate in the presence of acid.

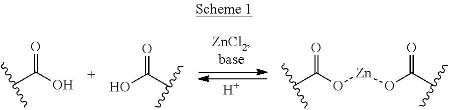

Scheme 1 above illustrates the reaction of an aqueous zinc ion (e.g. from zinc chloride) with two equivalents of an appropriate carboxylic acid to form the zinc dicarboxylate. This reaction occurs rapidly and irreversibly in a slightly basic pH environment but upon acidification, is reversible within a tunable range of pH 4.0-6.8 to reform ZnX₂, where X is the conjugate base. One of ordinary skill in the art will recognize that a variety of natural and unnatural amino acid side-chains have a carboxylic acid moiety that can be crosslinked by zinc or another suitable metal.

The choice of zinc as a crosslinking metal is advantageous for effective micelle crosslinking Zinc chloride and the zinc lactate by-product are generally recognized as non-toxic, and other safety concerns are not anticipated. Pharmaceutical grade zinc chloride is commonly used in mouthwash and as a chlorophyll stabilizer in vegetables while zinc lactate is used as an additive in toothpaste and drug preparation. While zinc has been chosen as an exemplary metal for micelle crosslinking, it should be noted that many other metals undergo acid sensitive coupling with imidazole derivatives. These metals include calcium, iron, copper, nickel and other transition metals. One or more of these metals can be substituted for zinc.

The ultimate goal of metal-mediated crosslinking is to ensure micelle stability when diluted in the blood (pH 7.4) followed by rapid dissolution and polynucleotide release in response to a finite pH change such as those found in tumor environments or in intracellular compartments. Previous reports suggest that the zinc-histidine bonds are stable above a threshold pH, below which dissociation to zinc ions and histidine occurs. (Tezcan, et. al. J. Am. Chem. Soc. 2007, 129, 13347-13375.)

One embodiment of the present invention utilizes the iron-mediated coupling of carboxylic acids to crosslink the micelle together. The interaction between iron and carboxylic acids in biological systems is well known in the art. (Silver, “Chemistry of Iron” 1993). Without wishing to be bound to any particular theory, it is believed that the carboxylic acid will function as a ligand in the carboxylate form (i.e. high pH) but will readily disassociate when the proton ion concentration is sufficiently high (i.e. low pH). (Scheme 3) In some embodiments, the iron moiety is Fe²⁺. In some embodiments, the iron moiety is Fe³⁺.

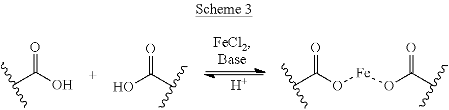

In one embodiment, drug-loaded micelles possessing carboxylic acid functionality in the outer core are crosslinked by addition of iron (II) chloride to the micelle solution. In another embodiment, drug-loaded micelles possessing carboxylic acid functionality in the outer core are crosslinked by addition of iron (III) chloride to the micelle. In certain embodiments, drug-loaded micelles possessing carboxylic acid functionality in the outer core are crosslinked by dissolving the micelles in TRIS buffer solution containing iron (II) chloride. In yet other embodiments, drug-loaded micelles possessing carboxylic acid functionality in the outer core are crosslinked by dissolving the micelles in TRIS buffer solution containing iron (III) chloride.

In certain embodiments, drug-loaded micelles possessing carboxylic acid functionality in the outer core are crosslinked by addition of iron (II) chloride to the micelle solution, followed by adjustment of the pH to 7-8. In other embodiments, drug-loaded micelles possessing carboxylic acid functionality in the outer core are crosslinked by addition of iron (III) chloride to the micelle solution, followed by adjustment of the pH to 7-8.

### Formula III

In certain embodiments, BH3-M6 loaded micelles of the present invention comprise a crosslinked multiblock polymer of formula III:

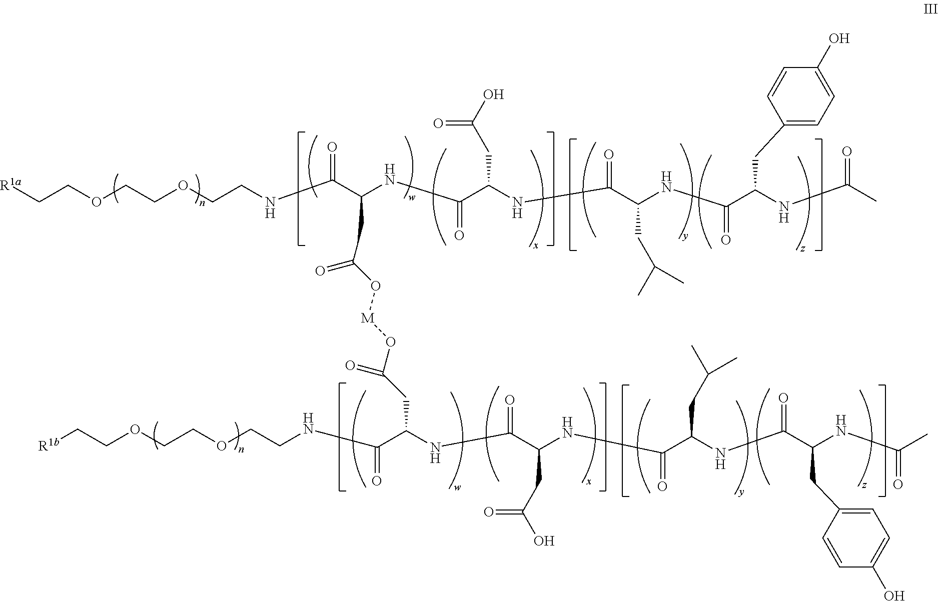

wherein:
R^{1a} and R^{1b} are independently selected from —OCH₃, —N₃,

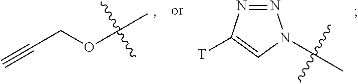

Wherein: T is a targeting group moiety;
M is a suitable metal ion;
n is 110 to 450;
w is 3 to 50;
x is 0 to 50, provided that the sum of w and x is no more than 50;
y is 5 to 50; and
z is 5 to 50.

As defined generally above, the n group of formula III is 110-450. In certain embodiments, the present invention provides compounds of formula III, as described above, wherein n is about 225. In other embodiments, n is about 270. In other embodiments, n is about 350. In other embodiments, n is about 110. In other embodiments, n is about 450. In other embodiments, n is selected from 110±10, 180±10, 225±10, 275±10, 315±10, or 450±10.

In certain embodiments, the w group of formula III is about 3 to about 50. In certain embodiments, the w group of formula III is 10. In other embodiments, w is about 5-10. According to yet another embodiment, w is about 1-10. In other embodiments, w is about 5. In other embodiments, w is selected from 5±3, 10±3, 10±5, 15±5, or 20±5.

In certain embodiments, the x group of formula III is about 0 to about 50. In certain embodiments, the x group of formula III is 0. In other embodiments, x is about 0-5. According to yet another embodiment, x is about 10. In other embodiments, x is about 5. In other embodiments, x is selected from 3±3, 5±3, 10±5, 15±5, or 20±5.

In certain embodiments, the y group of formula III is about 5 to about 50. In certain embodiments, the y group of formula III is about 10. In other embodiments, y is about 20. According to yet another embodiment, y is about 15. In other embodiments, y is about 30. In other embodiments, y is selected from 10±3, 15±3, 17±3, 20±5, 30±5, or 40±5.

In certain embodiments, the z group of formula III is about 5 to about 50. In certain embodiments, the z group of formula III is about 10. In other embodiments, z is about 20. According to yet another embodiment, z is about 15. In other embodiments, z is about 30. In other embodiments, z is selected from 10±3, 15±3, 17±3, 20±5, 30±5, or 40±5.

In some embodiments, the T targeting group moiety of formula III is a moiety selected from folate, a Her-2 binding peptide, a urokinase-type plasminogen activator receptor (uPAR) antagonist, a CXCR4 chemokine receptor antagonist, a GRP78 peptide antagonist, an RGD peptide, an RGD cyclic peptide, a luteinizing hormone-releasing hormone (LHRH) antagonist peptide, an aminopeptidase targeting peptide, a brain homing peptide, a kidney homing peptide, a heart homing peptide, a gut homing peptide, an integrin homing peptide, an angiogencid tumor endothelium homing peptide, an ovary homing peptide, a uterus homing peptide, a sperm homing peptide, a microglia homing peptide, a synovium homing peptide, a urothelium homing peptide, a prostate homing peptide, a lung homing peptide, a skin homing peptide, a retina homing peptide, a pancreas homing peptide, a liver homing peptide, a lymph node homing peptide, an adrenal gland homing peptide, a thyroid homing peptide, a bladder homing peptide, a breast homing peptide, a neuroblastoma homing peptide, a lymphoma homing peptide, a muscle homing peptide, a wound vasculature homing peptide, an adipose tissue homing peptide, a virus binding peptide, or a fusogenic peptide. Such targeting groups are well known in the art and are described in detail in WO 2008/134761.

In some embodiments, the T targeting group is a moiety selected from a tumor homing group, a prostate specific membrane antigen homing peptide, an aminopeptidate N homing peptide, a Her-2 homing peptide, a colon cancer homing peptide, a VEGFR1 homing peptide, or a CXCR4 homing peptide.

In certain embodiments, the -M- moiety of formula III is zinc. In another embodiment, the -M- moiety of formula III is iron. In other embodiments, M is selected from Ag, Fe, Cu, Ca, Mg, Ni, or Co. One of ordinary skill in the art will recognize that an BH3-M6 loaded micelle of formula III can be prepared from a mixture of Formula I and Formula II.

### General Methods for Providing Micelle Compounds Containing BH3-M6

Bifunctional PEG's are prepared according to U.S. Patent Application Publication Numbers 2006/0240092, 2006/0172914, 2006/0142506, and 2008/0035243, and Published PCT Applications WO07/127,473, WO07/127,440, and WO06/86325, the entirety of each of which is hereby incorporated by reference.

Multiblock copolymers of the present invention are prepared by methods known to one of ordinary skill in the art and those described in detail in U.S. patent application Ser. No. 11/325,020 filed Jan. 4, 2006 and published as U.S. 2006/0172914 on Aug. 3, 2006, the entirety of which is hereby incorporated herein by reference. Generally, such multiblock copolymers are prepared by sequentially polymerizing one or more cyclic amino acid monomers onto a hydrophilic polymer having a terminal amine salt wherein said polymerization is initiated by said amine salt. In certain embodiments, said polymerization occurs by ring-opening polymerization of the cyclic amino acid monomers. In other embodiments, the cyclic amino acid monomer is an amino acid NCA, lactam, or imide. Details of preparing exemplary multiblock copolymers of the present invention are set forth in the Examples.

Methods of preparing micelles are known to one of ordinary skill in the art. Micelles can be prepared by a number of different dissolution methods. In the direct dissolution method, the block copolymer is added directly to an aqueous medium with or without heating and micelles are spontaneously formed upon dissolution. The dialysis method is often used when micelles are formed from poorly aqueous soluble copolymers. The copolymer is dissolved in a water miscible organic solvent such as N-methyl pyrollidinone, dimethylformamide, dimethylsulfoxide, tetrahydrofuran, or dimethylacetamide, and this solution is then dialyzed against water or another aqueous medium. During dialysis, micelle formation is induced and the organic solvent is removed. Alternatively, the block copolymer can be dissolved in a water miscible organic solvent such as N-methylpyrollidinone, dimethylformamide, dimethylsulfoxide, tetrahydrofuran, or dimethylacetamide and added dropwise to water or another aqueous medium. The micelles can then be isolated by filtration or lyophilization.

Emulsification methods can also be employed for micelle formation. For example, the block copolymer is dissolved in a water-immiscible, volatile solvent (e.g. dichloromethane) and added to water with vigorous agitation. As the solvent is removed by evaporation, micelles spontaneously form. Prepared micelles can then be filtered and isolated by lyophilization.

Micelles can be prepared by a number of different dissolution methods. In the direct dissolution method, the block copolymer is added directly to an aqueous medium, with or without heating, and micelles are spontaneously formed up dissolution. The dialysis method is often used when micelles are formed from poorly aqueous soluble copolymers. The copolymer is dissolved in a water miscible organic solvent such as N-methylpyrollidinone, dimethylformamide, dimethylsulfoxide, tetrahydrofuran, or dimethylacetamide, and this solution is then dialyzed against water or another aqueous medium. During dialysis, micelle formation is induced and the organic solvent is removed. Alternatively, the block copolymer can be dissolved in a water miscible organic solvent such as N-methylpyrollidinone, dimethylformamide, dimethylsulfoxide, tetrahydrofuran, or dimethylacetamide and added dropwise to water or another aqueous medium. The micelles can then be isolated by filtration or lyophilization.

In one embodiment, drug-loaded micelles possessing carboxylic acid functionality in the outer core are crosslinked by addition of zinc chloride to the micelle solution along with a small amount of sodium hydroxide to neutralize any hydrochloric acid by-product. In this basic pH environment, the reaction of zinc chloride with the poly(aspartic acid) crosslinking block should be rapid and irreversible.

In another embodiment, drug loaded micelles possessing amine functionality in the outer core are crosslinked by the addition of a bifunctional, or multi-functional aldehyde-containing molecule which forms pH-reversible imine crosslinks. In another embodiment, drug loaded micelles possessing aldehyde functionality in the outer core are crosslinked by the addition of a bifunctional, or multi-functional amine-containing molecule which forms pH-reversible imine crosslinks.

In another embodiment, drug loaded micelles possessing alcohol or amine functionality in the outer core are crosslinked by the addition of a bifunctional, or multi-functional carboxylic acid-containing molecules and a coupling agent to form amide or ester crosslinks. In yet another embodiment, drug loaded micelles possessing carboxylic acid functionality in the outer core are crosslinked by the addition of a bifunctional, or multi-functional amine or alcohol-containing molecules and a coupling agent to form amide or ester crosslinks. Such coupling agents include, but are not limited to, carbodiimides (e.g. 1-ethyl-3-(3-dimethylaminopropyl)-carbodiimide (EDC), diisopropyl carbodiimide (DIC), dicyclohexyl carbodiimide (DCC)), aminium or phosphonium derivatives (e.g. PyBOP, PyAOP, TBTU, HATU, HBTU), or a combination of 1-hydroxybenzotriazole (HOBt) and an aminium or phosphonium derivative.

In another embodiment, drug loaded micelles possessing aldehyde or ketone functionality in the outer core are crosslinked by the addition of a bifunctional, or multifunctional hydrazine or hydrazide-containing molecule to form pH-reversible hydrazone crosslinks. In still other embodiments, drug loaded micelles hydrazine or hydrazide-functionality in the outer core are crosslinked by the addition of a bifunctional, or multifunctional aldehyde or ketone-containing molecule to form pH-reversible hydrazone crosslinks.

In another embodiment, drug loaded micelles possessing thiol functionality in the outer core are crosslinked by the addition of an oxidizing agent (e.g. metal oxides, halogens, oxygen, peroxides, ozone, peroxyacids, etc.) to form disulfide crosslinks. It will be appreciated that disulfide crosslinks are reversible in the presence of a suitable reducing agent (e.g. glutathione, dithiothreitol (DTT), etc.).

In yet another embodiment, drug loaded micelles possessing both carboxylic acid and thiol functionality in the outer core can be dual crosslinked by the addition of an oxidizing agent (e.g. metal oxides, halogens, oxygen, peroxides, ozone, peroxyacids, etc.) to form disulfide crosslinks followed by the addition of zinc chloride to the micelle solution along with a small amount of sodium bicarbonate to neutralize any hydrochloric acid by-product. It will be appreciated that such a dual-crosslinked micelle is reversible only in the presence of acid and a reducing agent (e.g. glutathione, dithiothreitol (DTT), etc.).

### Examples of BH3-M6 Encapsulated Micelles

### Example 1

### Encapsulation of BH3-M6

BH3-M6 (25 mg) was dissolved in 20% methanol indichloromethane (3 mL) and triblock co-polymer (500 mg, mPEG(12K)-block P(Asp)10-block-P(dLeu20-co-Tyr20)-Ac) was dissolved in water (300 mL). The drug containing organic phase was added the polymer aqueous solution while shear mixing at 10,000 RPM (Silverson mixer). The sample was allowed to mix at 10,000 RPM for approximately 30 seconds, then the resulting emulsion was allowed to stir at room temperature over night. Evaporation of the organic solvent over time results in encapsulation of the BH3-M6 in the core of micellar nanoparticles. The formulation is filtered through a 0.22 μm dead-end filter, frozen at −40° C., and stored as a dry powder. 450 mg was recovered for a 90% yield.

### Example 2

### Crosslinking of BH3-M6 Loaded Micelles

The powder from Example 1 was reconstituted in a 10 mM iron (III) chloride solution at 20 mg/mL, pH adjusted to 8.0 using 0.1N NaOH, and allowed to stir for approximately 16 hours to crosslink the formulation for stability. The formulation was then frozen at −40° C., and lyophilized.

### Example 3

### Weight Loading Determination of BH3-M6 Loaded Micelles

Following lyophilization the weight loading of the formulation was determined by HPLC analysis (Lichrosphere Select B column; isocratic mobile phase 85% methanol 15% 10 mM pH 2 phosphate buffer, fluorescence detection Ex 300 nm, Em 360 nm) by comparing the peak area of the formulation at 5 mg/mL to a standard curve of the free drug, both prepared in methanol as a diluent. The weight loading of the micelle Example 2 was found to be 2.8%.

### Example 4

### Particle Size Determination of BH3-M6 Loaded Micelles

Particle size was determined by a DynaPRO 96-well plate reader equipped with Dynamic Light Scattering. Briefly, the formulation was dissolved in 150 mM NaCl at 1 mg/mL, centrifuged at 2000 RPM for 5 minutes, and 300 μL aliquots were added to 3 separate wells. Particle size was determined by averaging 10 30-second acquisitions per well for 3 wells. The particle diameter of the micelle Example 2 was found to be 50 nM.

### Example 5

### Encapsulation Efficiency of BH3-M6 Loaded Micelles

Encapsulation dialysis was done by dissolving 4 mL of the formulation at 20 mg/mL in 10 mM phosphate buffer, and dialyzing 3 mL against 300 mL of the same phosphate buffer in 3500 molecular weight cut-off dialysis bags for 6 hours. The remaining 1 mL was used for the pre dialysis sample and after 6 hours the solution from inside the dialysis bag is taken for HPLC analysis. Peak areas from pre and post dialysis samples are used to determine the % BH3M6 remaining. The micelle from Example 1 was shown to have 88% of the drug retaining within the bag, indicating that the drug was effectively encapsulated within the micelle core.

### Example 6

### Crosslinking Efficiency of BH3-M6 Loaded Micelles

Crosslinking dialysis was performed by dissolving 4 mL of the formulation at 0.2 mg/mL in 10 mM phosphate buffer, and dialyzing 3 mL against 300 mL of the same phosphate buffer in 3500 molecular weight cut-off dialysis bags for 6 hours. The remaining 1 mL was used for the pre dialysis sample and after 6 hours the solution from inside the dialysis bag is taken for HPLC analysis. Peak areas from pre and post dialysis samples are used to determine the % BH3M6 remaining. The micelle from Example 1 was shown to have 80% of the drug retaining within the bag, indicating that the micelle was effectively crosslinked and is stable to dilution.

### Example 7

### In Vitro Efficacy of BH3-M6 Loaded Micelles

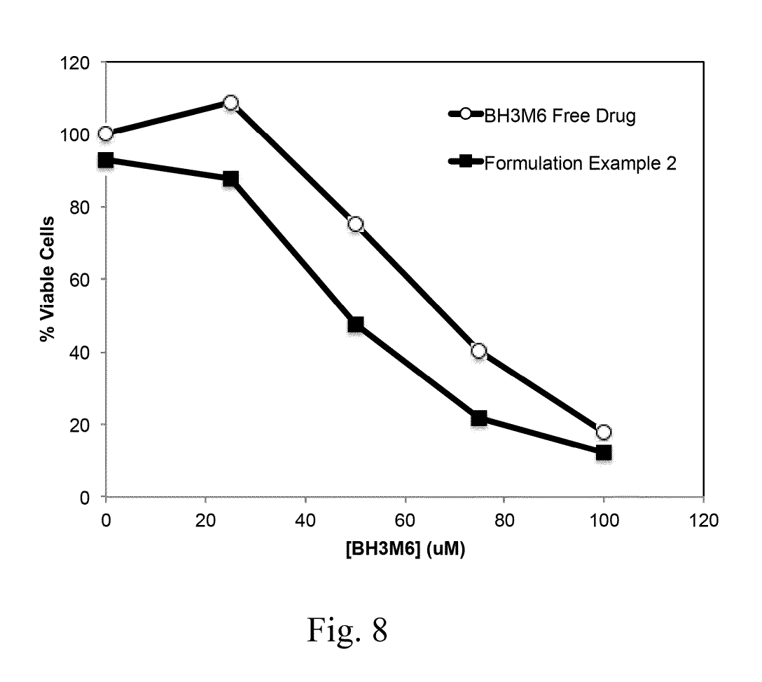

In-vitro cytotoxiciy was determined using A549 lung cancer cells exposed to a dose escalation of either BH3-M6 free drug in DMSO or the formulation from Example 2 dissolved in media in the range of about 0-100 μM BH3-M6. Briefly, cells were plated at approximately 50% confluency and allowed to attach over night. The next day the free drug dissolved in DMSO or the formulation dissolved in tissue culture media (Ham's F12K, 10% FBS, 1% Penicillin/Streptomycin) were added to the cells and left to incubate for 3 days. 72 hours later the cytotoxicity induced by the compounds was analyzed by Cell Titer-Glow live cell luminescence assay (Perkin-Elmer). The results of this assay are shown in FIG. 8.

### Example 8

### In Vivo Pharmacokinetics and Tumor Accumulation of BH3-M6 Loaded Micelles

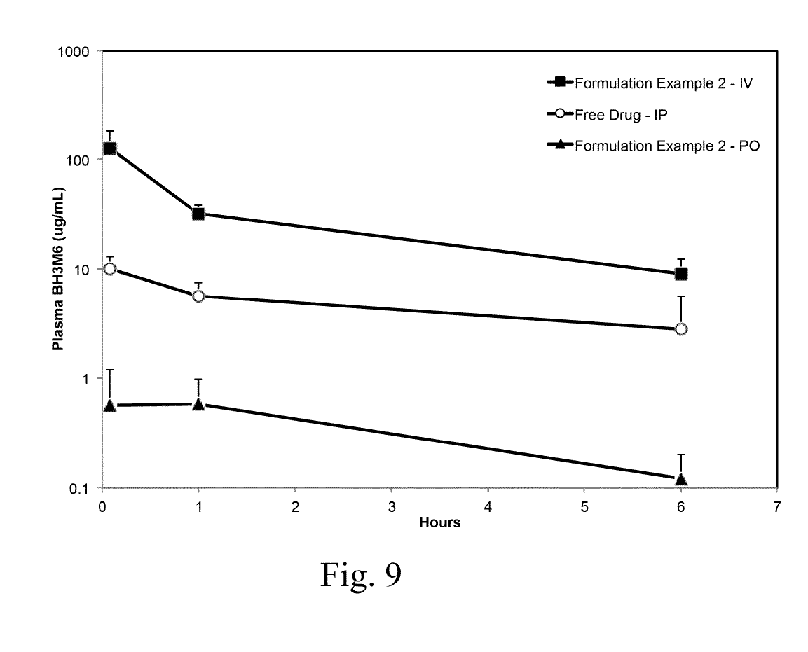

An A549 lung cancer xenograft model was used for the plasma and tumor accumulation studies. A549 lung cancer xenografts were grown in nude mice by implanting approximately 10 million cells into the right flank of each mouse, and allowed 3 weeks for the tumors to establish log growth and reach about 300-400 mm³. The formulation from Example 2 was dissolved in 150 mM NaCl for both intravenous (IV) administration and oral administration. The free drug was dissolved in DMSO for intraperitoneal (IP) administration. Time points for sample collection were 5 minutes, 1 hour and 6 hours post administration. Naive, tumor-bearing mice were used as controls for blank plasma and tumor tissue. Blood was collected by heart puncture, where approximately 500 μL was added to a 1.5 mL collection tube containing EDTA as anticoagulant. Plasma was isolated from blood by centrifugation at 2000 RPM for 5 minutes. The BH3-M6 compound was extracted from plasma by adding 50 μL of each plasma sample to 150 μL ice cold methanol. Samples were vortexed for 10 minutes, centrifuged at 13,000 RPM for 10 minutes, and the methanol supernatant was collected for HPLC analysis to determine plasma pharmacokinetics of the BH3-M6 compound. The results of this assay are shown in Table 1 and FIG. 9.

TABLE 1

| | AUC | | | |
|---|---|---|---|---|
| | (ug * hr/ | T½ | CMax | |
| Sample | mL) | (hr) | (ug) | TMax |
| Example | 177.7 | 1.8 | 129 | 5 min |
| 2, IV | | | | |
| Example | 2.3 | 2.5 | 0.58 | 1 hr |
| 2, Oral | | | | |
| Free drug, | 28.5 | 3.7 | 10.1 | 5 min |
| IP | | | | |

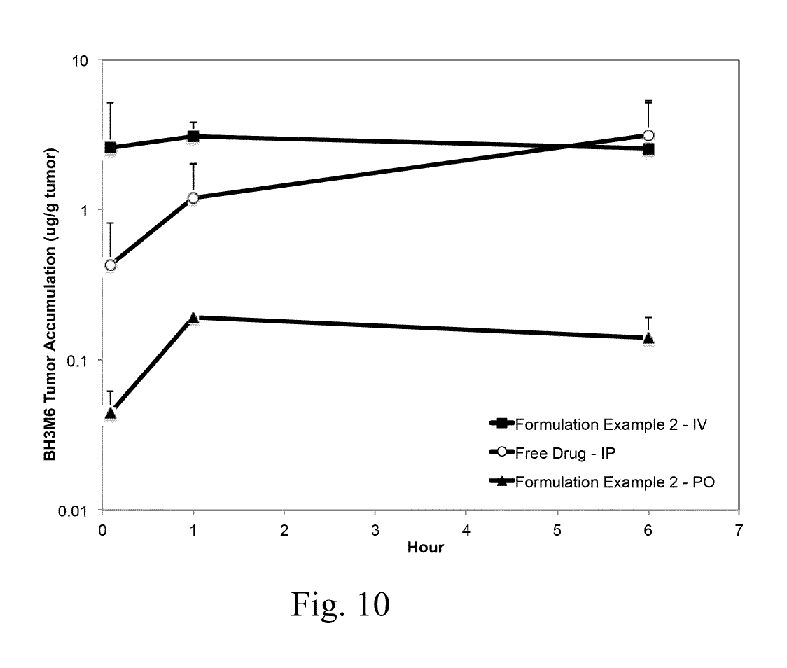

Tumor samples were taken from the mice after the blood samples, and snap frozen in liquid nitrogen until processed for HPLC analysis. The tumor samples were thawed, weighed and homogenized in 10 mM HEPES buffer. The tumor homogenate was processed as the plasma samples for HPLC analysis. The results of this assay are shown in Table 2 and FIG. 10.

TABLE 2

| | | CMax | |
|---|---|---|---|
| | AUC | (ug/g | |
| Sample | (ug * hr/g) | tumor) | TMax |
| Example 2, IV | 16.6 | 3.1 | 1 hr |
| Example 2, | 0.94 | 0.19 | 1 hr |
| Oral | | | |
| Free drug, IP | 11.5 | 3.1 | 6 hr |

In summary, a strategy of α-helix mimicry based on a substituted terphenyl scaffold was successfully applied to develop a pan Bcl-2 family antagonist. The disruption of protein-protein interactions between pro- and anti-apoptotic Bcl-2 family members with non-peptidic small molecules that mimic large areas of protein surfaces such as α-helices is a major milestone in the challenging field of protein-protein disruptor discovery. This is an important milestone as protein-protein interactions involving α-helices are implicated in many pathological conditions and are hence promising targets for drug discovery. Furthermore, the ability of BH3-M6 to disrupt interactions of anti-apoptotic proteins with both multi-domain and BH3-only pro-apoptotic proteins, its induction of Bax conformational change and its reliance on caspase and Bax for apoptosis induction suggest that the essential multi-domain pro-apoptotic proteins can be activated to induce apoptosis. These results, along with the fact that the ability of BH3-M6 to induce apoptosis is dependent on disrupting Bcl-X_{L}, Bcl-2 and Mcl-1 interactions with Bim, suggest that BH3-M6 induces apoptosis by the intended mechanism. The pan-Bcl-2 inhibitor BH3-M6 may be encapsulated in a micelle to provide a more bioavailable therapeutic agent. Specifically, the BH3-M6 compound may be encapsulated within a micelle comprising a multiblock copolymer according to the methods described herein. The compounds described herein provide a great advancement in cancer therapeutics used to treat cancers in which Bcl-2 is overexpressed.

The disclosures of all publications cited above are expressly incorporated herein by reference, each in its entirety, to the same extent as if each were incorporated by reference individually.

It is also to be understood that the following claims are intended to cover all of the generic and specific features of the invention herein described, and all statements of the scope of the invention which, as a matter of language, might be said to fall there between. Now that the invention has been described,

What is claimed is:

1. A compound comprised of the following chemical structure: 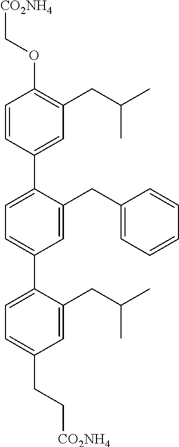
or pharmaceutically acceptable salts, prodrugs, salts of a prodrugs and metabolites thereof.

2. A micelle comprising a multiblock copolymer of formula I: 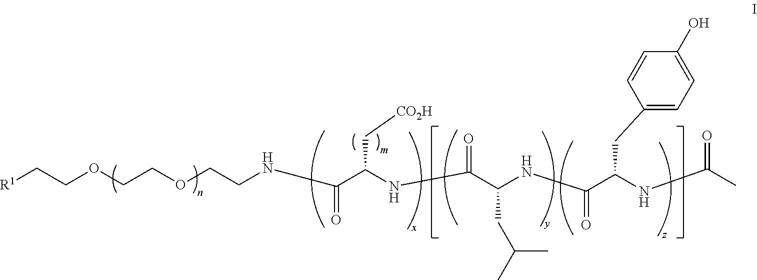
wherein:
R¹ is —OCH₃, —N₃, or 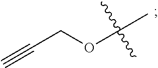 [SMILES: *OCC#C]
n is 110 to 450;
m is 1 or 2;
x is 3 to 50;
y is 5 to 50; and
z is 5 to 50;
wherein the micelle encapsulates BH3-M6 or pharmaceutically acceptable salts, prodrugs, salts of a prodrugs and metabolites thereof.

3. The micelle of claim 2, wherein R¹ is —N₃.

4. The micelle of claim 2, wherein R¹ is —CH₃.

5. A micelle comprising a crosslinked multiblock polymer of formula III: 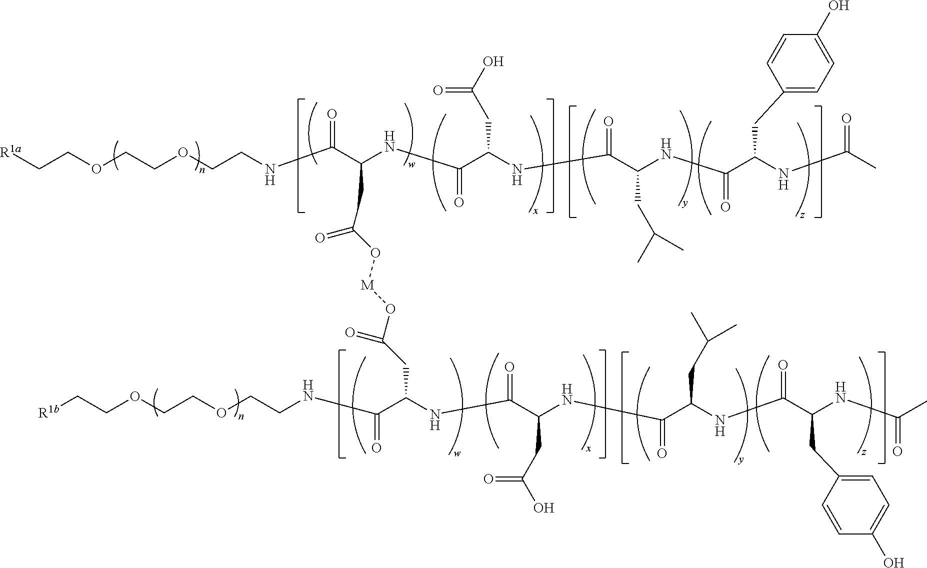
III
wherein:
R^{1a} and R^{ib} are independently selected from —OCH₃, —N₃, 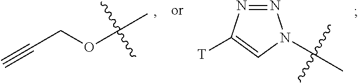
T is a targeting group moiety;
M is a metal ion;
n is 110 to 450;
w is 3 to 50;
x is 0 to 50, provided that the sum of w and x is no more than 50;
y is 5 to 50; and
z is 5 to 50;
wherein the micelle encapsulates BH3-M6 or pharmaceutically acceptable salts, prodrugs, salts of a prodrugs and metabolites thereof.

6. The micelle of claim 5, wherein R^{1a} and R^{1b} are both —CH₃.

7. The micelle of claim 5, wherein M is iron.

8. A method of treating cancer comprising administering a therapeutically effective amount of at least one pan-Bcl-2 inhibitor to a subject in need thereof wherein the at least one pan-Bcl-2 inhibitor is BH3-M6 or pharmaceutically acceptable salts, prodrugs, salts of a prodrugs and metabolites thereof.

9. The method of claim 8, wherein the pan-Bcl-2 inhibitor is encapsulated in a multiblock copolymer of Formula I.

10. The method of claim 8, wherein the pan-Bcl-2 inhibitor is encapsulated in a crosslinked multiblock polymer of Formula III.

11. The method of claim 8, wherein the cancer is characterized by the overexpression of an anti-apoptotic Bcl-2 family protein.

12. The method of claim 8, wherein the cancer is selected from the group consisting of breast cancer, non-small cell lung cancer, prostate cancer and adenocarcinoma.

13. The method of claim 8, further comprising administering at least one active agent.

14. The method of claim 13, wherein the active agent is a proteasome inhibitor.

15. The method of claim 14, wherein the proteasome inhibitor is selected from the group consisting of CEP-1612, bortezomib, NPI-0052, PR-171 and MG-132.

16. A method of inducing apoptosis in a cancer cell comprising administering at least one pan-Bcl-2 inhibitor to the cell wherein the at least one pan-Bcl-2 inhibitor is BH3-M6 or pharmaceutically acceptable salts, prodrugs, salts of a prodrugs and metabolites thereof.

17. The method of claim 16, wherein the pan-Bcl-2 inhibitor is encapsulated in a multiblock copolymer of Formula I.

18. The method of claim 16, wherein the pan-Bcl-2 inhibitor is encapsulated in a crosslinked multiblock polymer of Formula III.

19. The method of claim 16, further comprising administering at least one active agent.

20. The method of claim 19, wherein the active agent is a proteasome inhibitor.

21. The method of claim 20, wherein the proteasome inhibitor is selected from the group consisting of CEP-1612, bortezomib, NPI-0052, PR-171 and MG-132.

22. A composition for treating cancer comprising:
at least one pan-Bcl-2 inhibitor wherein the at least one pan-Bcl-2 inhibitor is BH3-M6 or pharmaceutically acceptable salts, prodrugs, salts of a prodrugs and metabolites thereof; and
a pharmaceutically acceptable carrier.

23. The method of claim 22, wherein the pharmaceutically acceptable carrier is a micelle multiblock copolymer of Formula I.

24. The method of claim 23, wherein the at least one pan-Bcl-2 inhibitor is encapsulated in the micelle multiblock copolymer of Formula I.

25. The method of claim 22, wherein the pharmaceutically acceptable carrier is a micelle crosslinked multiblock polymer of Formula III.

26. The method of claim 25, wherein the at least one pan-Bcl-2 inhibitor is encapsulated in the micelle crosslinked multiblock polymer of Formula III.

27. The method of claim 22, wherein the cancer is characterized by the overexpression of an anti-apoptotic Bcl-2 family protein.

28. The method of claim 22, wherein the cancer is selected from the group consisting of breast cancer, non-small cell lung cancer, prostate cancer and adenocarcinoma.

29. The method of claim 22, further comprising administering at least one active agent.

30. The method of claim 29, wherein the active agent is a proteasome inhibitor.

31. The method of claim 30, wherein the proteasome inhibitor is selected from the group consisting of CEP-1612, bortezomib, NPI-0052, PR-171 and MG-132.
